SEQUENCE LISTING (55 sequences)

SEQ ID NO: 1 - protein, 10 aa, Homo sapiens
SLAIGGVTEA

SEQ ID NO: 2 - protein, 11 aa, Homo sapiens
LLLGIAKLLKV

SEQ ID NO: 3 - protein, 9 aa, Homo sapiens
LLAEQPDQV

SEQ ID NO: 4 - protein, 9 aa, Homo sapiens
STMYYLWML

SEQ ID NO: 5 - protein, 114 aa, Homo sapiens
AQKVTQAQTEISVVEKEDVTLDCVYETRDTTYYLFWYKQPPSGELVFLIRRNSFDEQNEISGRYSWNFQKSTSSFNFTITASQVVDSAVYFCALSGGGFKTIFGAGTRLFVKAN

SEQ ID NO: 6 - protein, 9 aa, Homo sapiens
FLSELEPPA

SEQ ID NO: 7 - protein, 11 aa, Homo sapiens
FDLAYGSVITV

SEQ ID NO: 8 - protein, 9 aa, Homo sapiens
SLDGTTTKA

SEQ ID NO: 9 - DNA, 339 bp, Homo sapiens
  source 1..339: mol_type = genomic DNA
cttgctaagaccacccagcccatctccatggactcatatgaaggacaagaagtgaacataacctgtagccacaacaacattgctacaaatgattatatcacgtggtaccaacagtttcccagccaaggaccacgatttattattcaaggatacaagacaaaagttacaaacgaagtggcctccctgtttatccctgccgacagaaagtccagcactctgagcctgccccgggtttccctgagcgacactgctgtgtactactgcctcgtgggttggttctctggtggctacaataagctgatttttggagcagggaccaggctggctgtacacccatat

SEQ ID NO: 10 - DNA, 353 bp, Homo sapiens
  source 1..353: mol_type = genomic DNA
ggtgctgtcgtctctcaacatccgagcagggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagcgctaggggggcaggacaagagacccagtacttcgggccaggcacgcggctcctggtgctcgaggacct

SEQ ID NO: 11 - protein, 113 aa, Homo sapiens
LAKTTQPISMDSYEGQEVNITCSHNNIATNDYITWYQQFPSQGPRFIIQGYKTKVTNEVASLFIPADRKSSTLSLPRVSLSDTAVYYCLVGWFSGGYNKLIFGAGTRLAVHPY

SEQ ID NO: 12 - protein, 118 aa, Homo sapiens
GAVVSQHPSRVICKSGTSVKIECRSLDFQATTMFWYRQFPKQSLMLMATSNEGSKATYEQGVEKDKFLINHASLTLSTLTVTSAHPEDSSFYICSARGAGQETQYFGPGTRLLVLEDL

SEQ ID NO: 13 - DNA, 345 bp, Homo sapiens
  source 1..345: mol_type = genomic DNA
gctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcttataggagcgattccgggtatgcactcaacttcggcaaaggcacctcgctgttggtcacaccccat

SEQ ID NO: 14 - DNA, 351 bp, Homo sapiens
  source 1..351: mol_type = genomic DNA
gctgctggagtcatccagtccccaagacatctgatcaaagaaaagagggaaacagccactctgaaatgctatcctatccctagacacgacactgtctactggtaccagcagggtccaggtcaggacccccagttcctcatttcgttttatgaaaagatgcagagcgataaaggaagcatccctgatcgattctcagctcaacagttcagtgactatcattctgaactgaacatgagctccttggagctgggggactcagccctgtacttctgtgccagcagccccgggactagcggggggacagatacgcagtattttggcccaggcacccggctgacagtgctcggacct

SEQ ID NO: 15 - protein, 93 aa, Homo sapiens
AQTVTQSQPEMSVQEAETVTLSCTYDTSESDYYLFWYKQPPSRQMILVIRQEAYKQQNATENRFSVNFQKAAKSFSLKISDSQLGDAAMYFCA

SEQ ID NO: 16 - protein, 117 aa, Homo sapiens
AAGVIQSPRHLIKEKRETATLKCYPIPRHDTVYWYQQGPGQDPQFLISFYEKMQSDKGSIPDRFSAQQFSDYHSELNMSSLELGDSALYFCASSPGTSGGTDTQYFGPGTRLTVLGP

SEQ ID NO: 17 - DNA, 345 bp, Homo sapiens
  source 1..345: mol_type = genomic DNA
cagaaggaggtggagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtgaaacggtggtcaggaggaggtgctgacggactcacctttggcaaagggactcatctaatcatccagccctat

SEQ ID NO: 18 - DNA, 344 bp, Homo sapiens
  source 1..344: mol_type = genomic DNA
gatgccatggtcatccagaacccaagataccaggttacccagtttggaaagccagtgaccctgagttgttctcagactttgaaccataacgtcatgtactggtaccagcagaagtcaagtcaggccccaaagctgctgttccactactatgacaaagattttaacaatgaagcagacacccctgataacttccaatccaggaggccgaacacttctttctgctttcttgacatccgctcaccaggcctgggggacgcagccatgtacctgtgtgccaccagcagagttcggggcactgaagctttctttggacaaggcaccagactcacagttgtagaggacct

SEQ ID NO: 19 - protein, 115 aa, Homo sapiens
QKEVEQNSGPLSVPEGAIASLNCTYSDRGSQSFFWYRQYSGKSPELIMSIYSNGDKEDGRFTAQLNKASQYVSLLIRDSQPSDSATYLCAVKRWSGGGADGLTFGKGTHLIIQPY

SEQ ID NO: 20 - protein, 115 aa, Homo sapiens
DAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSRVRGTEAFFGQGTRLTVVEDL

SEQ ID NO: 21 - DNA, 330 bp, Homo sapiens
  source 1..330: mol_type = genomic DNA
ggacaaagccttgagcagccctctgaagtgacagctgtggaaggagccattgtccagataaactgcacgtaccagacatctgggttttatgggctgtcctggtaccagcaacatgatggcggagcacccacatttctttcttacaatggtctggatggtttggaggagacaggtcgtttttcttcattccttagtcgctctgatagttatggttacctccttctacaggagctccagatgaaagactctgcctcttacttctgcgctgtagacaccaatgcaggcaaatcaacctttggggatgggactacgctcactgtgaagccaaat

SEQ ID NO: 22 - DNA, 353 bp, Homo sapiens
  source 1..353: mol_type = genomic DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtacttccgggacagggctatcgtcacccctccactttgggaacgggaccaggctcactgtgacagaggacct

SEQ ID NO: 23 - protein, 110 aa, Homo sapiens
GQSLEQPSEVTAVEGAIVQINCTYQTSGFYGLSWYQQHDGGAPTFLSYNGLDGLEETGRFSSFLSRSDSYGYLLLQELQMKDSASYFCAVDTNAGKSTFGDGTTLTVKPN

SEQ ID NO: 24 - protein, 118 aa, Homo sapiens
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSTSGTGLSSPLHFGNGTRLTVTEDL

SEQ ID NO: 25 - DNA, 342 bp, Homo sapiens
  source 1..342: mol_type = genomic DNA
cagaaggaggtggagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtgcccgctggtggtactagctatggaaagctgacatttggacaagggaccatcttgactgtccatccaaat

SEQ ID NO: 26 - DNA, 341 bp, Homo sapiens
  source 1..341: mol_type = genomic DNA
aaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagcccgacagggactgaagctttctttggacaaggcaccagactcacagttgtagaggacct

SEQ ID NO: 27 - protein, 114 aa, Homo sapiens
QKEVEQNSGPLSVPEGAIASLNCTYSDRGSQSFFWYRQYSGKSPELIMSIYSNGDKEDGRFTAQLNKASQYVSLLIRDSQPSDSATYLCAVPAGGTSYGKLTFGQGTILTVHPN

SEQ ID NO: 28 - protein, 114 aa, Homo sapiens
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSPTGTEAFFGQGTRLTVVEDL

SEQ ID NO: 29 - DNA, 339 bp, Homo sapiens
  source 1..339: mol_type = genomic DNA
aaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgacccgtattaacgactacaagctcagctttggagccggaaccacagtaactgtaagagcaaat

SEQ ID NO: 30 - DNA, 338 bp, Homo sapiens
  source 1..338: mol_type = genomic DNA
aaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagctcaatgaacactgaagctttctttggacaaggcaccagactcacagttgtagaggacct

SEQ ID NO: 31 - protein, 113 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVTRINDYKLSFGAGTTVTVRAN

SEQ ID NO: 32 - protein, 113 aa, Homo sapiens
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSMNTEAFFGQGTRLTVVEDL

SEQ ID NO: 33 - DNA, 336 bp, Homo sapiens
  source 1..336: mol_type = genomic DNA
gcccagtctgtgagccagcataaccaccacgtaattctctctgaagcagcctcactggagttgggatgcaactattcctatggtggaactgttaatctcttctggtatgtccagtaccctggtcaacaccttcagcttctcctcaagtacttttcaggggatccactggttaaaggcatcaagggctttgaggctgaatttataaagagtaaattctcctttaatctgaggaaaccctctgtgcagtggagtgacacagctgagtacttctgtgccgcgtataacaccgacaagctcatctttgggactgggaccagattacaagtctttccaaat

SEQ ID NO: 34 - DNA, 350 bp, Homo sapiens
  source 1..350: mol_type = genomic DNA
aaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagcggcgcggggggaccccaagagacccagtacttcgggccaggcacgcggctcctggtgctcgaggacct

SEQ ID NO: 35 - protein, 112 aa, Homo sapiens
AQSVSQHNHHVILSEAASLELGCNYSYGGTVNLFWYVQYPGQHLQLLLKYFSGDPLVKGIKGFEAEFIKSKFSFNLRKPSVQWSDTAEYFCAAYNTDKLIFGTGTRLQVFPN

SEQ ID NO: 36 - protein, 117 aa, Homo sapiens
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSGAGGPQETQYFGPGTRLLVLEDL

SEQ ID NO: 37 - DNA, 330 bp, Homo sapiens
  source 1..330: mol_type = genomic DNA
cagaaggaggtggagcaggatcctggaccactcagtgttccagagggagccattgtttctctcaactgcacttacagcaacagtgcttttcaatacttcatgtggtacagacagtattccagaaaaggccctgagttgctgatgtacacatactccagtggtaacaaagaagatggaaggtttacagcacaggtcgataaatccagcaagtatatctccttgttcatcagagactcacagcccagtgattcagccacctacctctgtgcaatgacaggaaacacacctcttgtctttggaaagggcacaagactttctgtgattgcaaat

SEQ ID NO: 38 - DNA, 347 bp, Homo sapiens
  source 1..347: mol_type = genomic DNA
ggtgctgtcgtctctcaacatccgagcagggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctttacagggcgctgaagctttctttggacaaggcaccagactcacagttgtagaggacct

SEQ ID NO: 39 - protein, 110 aa, Homo sapiens
QKEVEQDPGPLSVPEGAIVSLNCTYSNSAFQYFMWYRQYSRKGPELLMYTYSSGNKEDGRFTAQVDKSSKYISLFIRDSQPSDSATYLCAMTGNTPLVFGKGTRLSVIAN

SEQ ID NO: 40 - protein, 116 aa, Homo sapiens
GAVVSQHPSRVICKSGTSVKIECRSLDFQATTMFWYRQFPKQSLMLMATSNEGSKATYEQGVEKDKFLINHASLTLSTLTVTSAHPEDSSFYICSALQGAEAFFGQGTRLTVVEDL

SEQ ID NO: 41 - DNA, 342 bp, Homo sapiens
  source 1..342: mol_type = genomic DNA
ggagagagtgtggggctgcatcttcctaccctgagtgtccaggagggtgacaactctattatcaactgtgcttattcaaacagcgcctcagactacttcatttggtacaagcaagaatctggaaaaggtcctcaattcattatagacattcgttcaaatatggacaaaaggcaaggccaaagagtcaccgttttattgaataagacagtgaaacatctctctctgcaaattgcagctactcaacctggagactcagctgtctacttttgtgcagagaggggtggtactagctatggaaagctgacatttggacaagggaccatcttgactgtccatccaaat

SEQ ID NO: 42 - DNA, 222 bp, Homo sapiens
  source 1..222: mol_type = genomic DNA
gacacagctgtttcccagactccaaaatacctggtcacacagatgggaaacgacaagtccattaaatgtgaacaaaatctgggccatgatactatgtattggtataaacaggactctaagaaatttctgaagataatgtttagctacaataataaggagctcattataaatgaaacagttccaaatcgcttctcacctaaatctccagacaaagctcactta

SEQ ID NO: 43 - protein, 114 aa, Homo sapiens
GESVGLHLPTLSVQEGDNSIINCAYSNSASDYFIWYKQESGKGPQFIIDIRSNMDKRQGQRVTVLLNKTVKHLSLQIAATQPGDSAVYFCAERGGTSYGKLTFGQGTILTVHPN

SEQ ID NO: 44 - protein, 74 aa, Homo sapiens
DTAVSQTPKYLVTQMGNDKSIKCEQNLGHDTMYWYKQDSKKFLKIMFSYNNKELIINETVPNRFSPKSPDKAHL

SEQ ID NO: 45 - DNA, 336 bp, Homo sapiens
  source 1..336: mol_type = genomic DNA
cagaaggaggtggagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtgaatcatcagggagcccagaagctggtatttggccaaggaaccaggctgactatcaacccaaat

SEQ ID NO: 46 - DNA, 345 bp, Homo sapiens
  source 1..345: mol_type = genomic DNA
gattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcgtggggacccttaatgagcagttcttcgggccagggacacggctcaccgtgctagaggacctg

SEQ ID NO: 47 - protein, 112 aa, Homo sapiens
QKEVEQNSGPLSVPEGAIASLNCTYSDRGSQSFFWYRQYSGKSPELIMSIYSNGDKEDGRFTAQLNKASQYVSLLIRDSQPSDSATYLCAVNHQGAQKLVFGQGTRLTINPN

SEQ ID NO: 48 - protein, 115 aa, Homo sapiens
DSGVTQTPKHLITATGQRVTLRCSPRSGDLSVYWYQQSLDQGLQFLIQYYNGEERAKGNILERFSAQQFPDLHSELNLSSLELGDSALYFCASSVGTLNEQFFGPGTRLTVLEDL

SEQ ID NO: 49 - DNA, 336 bp, Homo sapiens
  source 1..336: mol_type = genomic DNA
acccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcggcccagcctggtgatacaggcctctacctctgtgcaggagtaccaggaggaagctacatacctacatttggaagaggaaccagccttattgttcatccgtat

SEQ ID NO: 50 - DNA, 357 bp, Homo sapiens
  source 1..357: mol_type = genomic DNA
gacacagctgtttcccagactccaaaatacctggtcacacagatgggaaacgacaagtccattaaatgtgaacaaaatctgggccatgatactatgtattggtataaacaggactctaagaaatttctgaagataatgtttagctacaataataaggagctcattataaatgaaacagttccaaatcgcttctcacctaaatctccagacaaagctcacttaaatcttcacatcaattccctggagcttggtgactctgctgtgtatttctgtgccagcagccaggtcgagacggaaggagctaactatggctacaccttcggttcggggaccaggttaaccgttgtagaggacctg

SEQ ID NO: 51 - protein, 116 aa, Homo sapiens
SQTIHQWPATLVQPVGSPLSLECTVEGTSNPNLYWYRQAAGRGLQLLFYSVGIGQISSEVPQNLSASRPQDRQFILSSKKLLLSDSGFYLCAWNGQGSYNEQFFGPGTRLTVLEDL

SEQ ID NO: 52 - protein, 112 aa, Homo sapiens
TQLLEQSPQFLSIQEGENLTVYCNSSSVFSSLQWYRQEPGEGPVLLVTVVTGGEVKKLKRLTFQFGDARKDSSLHITAAQPGDTGLYLCAGVPGGSYIPTFGRGTSLIVHPY

SEQ ID NO: 53 - protein, 119 aa, Homo sapiens
DTAVSQTPKYLVTQMGNDKSIKCEQNLGHDTMYWYKQDSKKFLKIMFSYNNKELIINETVPNRFSPKSPDKAHLNLHINSLELGDSAVYFCASSQVETEGANYGYTFGSGTRLTVVEDL

SEQ ID NO: 54 - DNA, 342 bp, Homo sapiens
  source 1..342: mol_type = genomic DNA
gctcagaaggtaactcaagcgcagactgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgagtggtggaggcttcaaaactatctttggagcaggaacaagactatttgttaaagcaaat

SEQ ID NO: 55 - DNA, 348 bp, Homo sapiens
  source 1..348: mol_type = genomic DNA
tctcagactattcatcaatggccagcgaccctggtgcagcctgtgggcagcccgctctctctggagtgcactgtggagggaacatcaaaccccaacctatactggtaccgacaggctgcaggcaggggcctccagctgctcttctactccgttggtattggccagatcagctctgaggtgccccagaatctctcagcctccagaccccaggaccggcagttcatcctgagttctaagaagctccttctcagtgactctggcttctatctctgtgcctggaacggacagggctcctacaatgagcagttcttcgggccagggacacggctcaccgtgctagaggacctg

(19) United States
(12) Patent Application Publication
(10) Pub. No.: US 2026/0285935 A1
(43) Pub. Date: Sep. 24, 2026

(54) HUMAN T CELL RECEPTORS TARGETING TUMOR-ENHANCED SPLICING EPITOPES ON HLA-A*02:01 FOR SMALL CELL CARCINOMA

(71) Applicants: The Regents of the University of California, Oakland, CA (US); THE CHILDREN’S HOSPITAL OF PHILADELPHIA, Philadelphia, PA (US)

(72) Inventors: Owen N. Witte, Sherman Oaks, CA (US); Yi Xing, Merion Station, PA (US); John W. Phillips; Jami McLaughlin Witte, Sherman Oaks, CA (US); Yang Pan, Chicago, IL (US); Miyako Noguchi, Culver City, CA (US); Zhiyuan Mao, Los Angeles, CA (US); Beatrice Zhang, New Orleans, LA (US)

(73) Assignees: The Regents of the University of California, Oakland, CA (US); THE CHILDREN’S HOSPITAL OF PHILADELPHIA, Philadelphia, PA (US)

(21) Appl. No.: 19/473,788

(22) PCT Filed: Apr. 11, 2024
(86) PCT No.: PCT/US2024/024049

§ 371 Date: Oct. 8, 2025

Related U.S. Application Data
(60) Provisional application No. 63/495,405, filed on Apr. 11, 2023.

(51) Int. Cl. C07K 14/705; A61K 38/00; A61K 40/11; A61K 40/32; A61K 40/42; A61P 35/00; C12N 5/0783; C12N 15/86

(52) U.S. Cl. CPC C07K 14/7051; A61K 40/11; A61K 40/32; A61K 40/421; A61P 35/00; C12N 5/0636; C12N 15/86; A61K 38/00; C12N 2510/00; C12N 2740/10043

(57) ABSTRACT

We utilized a newly developed computational method, Isoform peptides from RNA splicing for Immunotherapy target Screening methodology (IRIS), to process and capture small cell carcinoma-specific RNA splicing events. Candidate epitopes were used to stimulate peripheral mononuclear cells (PBMCs) from healthy donors. Reactive T cells were then isolated by either surface activation marker (CD137) or intracellular activation markers (IFNγ and TNFα). T cell receptors (TCRs) from recovered cells were retrieved by 10X Genomics single cell V(D)J sequencing. Paired TCR alpha and beta chain were engineered and introduced into normal human PBMCs and tested for functionality. We were able to define a group of TCRs targeting 7 distinct small cell cancer-enhanced splicing epitopes that predicted by our in-silico platform. Those TCRs specifically and efficiently recognize their cognate targets on HLA-A*02:01. These inventions can be used to develop new treatment for pan-small cell cancer treatments.

### CROSS REFERENCE TO RELATED APPLICATIONS

This application claims the benefit under 35 U.S.C. Section 119 (e) of co-pending and commonly-assigned U.S. Provisional Patent Application No. 63/495,405, filed Apr. 11, 2023, entitled “HUMAN T CELL RECEPTORS TARGETING TUMOR-ENHANCED SPLICING EPITOPES ON HLA-A*02:01 FOR SMALL CELL CARCINOMA”, the contents of which is incorporated by reference herein.

### STATEMENT REGARDING FEDERAL FUNDING

This invention was made with government support under Grant Number CA233074, awarded by the National Institutes of Health. The government has certain rights in the invention.

### TECHNICAL FIELD

Embodiments of the disclosure concern at least the fields of medicine and immunology.

### BACKGROUND OF THE INVENTION

The αβ T cell receptor (TCR) determines the unique specificity of each naïve T cell. Upon assembly with CD3 signaling proteins on the T cell surface, the TCR surveils peptide ligands presented by major histocompatibility complex (MHC) molecules on the surface of nucleated cells. The specificity of the TCR for a peptide-MHC complex is determined by both the presenting MHC molecule and the presented peptide. The MHC locus (also known as the human leukocyte antigen (HLA) locus in humans) is the most multi-allelic locus in the human genome, comprising >18,000 MHC class I and II alleles that vary widely in frequency across ethnic subgroups. Ligands presented by MHC class I molecules are derived primarily from proteasomal cleavage of endogenously expressed antigens. Infected and cancerous cells present peptides that are recognized by CD8+ T cells as foreign or aberrant, resulting in T cell-mediated killing of the presenting cell.

T cells can be engineered to kill tumor cells through the transfer of tumor-reactive αβ TCR genes. Key to this approach is that the patient expresses the MHC allele on which the therapeutic TCR is restricted and that the targeted peptide is derived from a tumor-associated or tumor-specific antigen. Private (patient-specific) neoantigens resulting from tumor-specific mutations are a potential source of such targets (4). However, implementation of personalized TCR gene therapy is complicated by the need to identify mutations through sequencing, to isolate mutation-reactive, patient-specific TCRs, and to genetically modify patient T cells on-demand. This is still more challenging for tumors that cannot be accessed for sequencing and for low mutational burden tumors with few or no neoantigens. Particularly for these last tumor types, targeting public (non-patient specific), tumor-restricted antigens with off-the-shelf TCRs remains an attractive option.

The first public antigen targeted with TCR gene therapy in the clinic was melanocyte antigen MARTI/Melan-A, yielding objective responses in 2/15 patients with metastatic melanoma. Use of a higher affinity MARTI-reactive TCR (F5) increased the response rate to 30% but also produced a variety of side effects including vitiligo, uveitis, and transient hearing loss due to MARTI expression on healthy melanocytes in the skin, eye, and middle ear. T cell therapies targeting other public antigens have similarly resulted in morbidity or other serious adverse events due to on-target/off-tumor reactivity. For example, targeting carcinoembryonic antigen produces severe colitis in patients with metastatic colorectal cancer due to reactivity with normal colorectal tissue. More seriously, T cell therapies targeted at ERBB2 or MAGE-A3 each resulted in deaths due to unappreciated expression of the target antigen (or similar variant) on vital organs. Thus, these studies underscore the importance of identifying stringently tumor-specific public antigens, particularly when well-expressed, high-affinity targeting receptors necessary for therapeutic success are employed.

Small cell carcinoma is a highly aggressive form of tumor commonly arising from epithelial cancers with no effective treatment. Different small cell cancers, such as small cell prostate cancer (SCPC) and small cell lung cancer (SCLC), are known to share convergent phenotypes. Pan-small cell targets can be utilized to target larger cohort of patients. Aberrant alternative splicing (AS) is prevalent in cancer, generating an extensive but largely unexplored repertoire of novel immunotherapy targets. Cancer-enhanced splicing variants are known to generate tumor-specific antigens and thus can be targeted by cell-mediated immunotherapies. However, as there is no widely accepted platform to identify those events, cell-mediated immunotherapies that can target cancer-enhanced splicing variants remain elusive.

For the reasons noted above, there is a need in the art for additional methods and materials useful for cell-mediated immunotherapies.

### SUMMARY OF THE INVENTION

As disclosed herein, we describe Isoform peptides from RNA splicing for Immunotherapy target Screening (IRIS), a computational platform capable of discovering AS-derived tumor antigens (TAs) for T-cell receptor (TCR) therapies. IRIS leverages large-scale data on tumor and normal transcriptomes and incorporates screening and prediction approaches to discover AS-derived TAs with tumor-associated or tumor-specific expression. In an exemplary analysis integrating transcriptomics and immunopeptidomics data, we showed that hundreds of IRIS-predicted TCR targets are presented by human leukocyte antigen (HLA) molecules. In one embodiment of this method, we applied IRIS to RNA-seq data of neuroendocrine prostate cancer (NEPC). From 2,939 NEPC-associated AS events, IRIS predicted 1,651 epitopes from 808 events as potential TCR targets for two common HLA types (A*02:01 and *A03:01). Predicted epitopes are often encoded by microexons of length≤30 nucleotides. To validate the immunogenicity and T-cell recognition of IRIS-predicted TCR epitopes, we performed an in vitro T-cell priming assay in combination with single-cell TCR sequencing. Multiple TCRs identified in this methodology were then transduced into human peripheral blood mononuclear cells. These TCR embodiments of the invention showed high activity against individual IRIS-predicted epitopes, providing strong evidence of isolated TCRs reactive to AS-derived peptides. Certain of these TCR embodiments showed efficient cytotoxicity against target cells expressing the target peptide. Our study illustrates the contribution of AS to the TA repertoire of cancer cells, and demonstrates the utility of IRIS for discovering novel TAs and expanding cancer immunotherapies.

As disclosed below, using the Immunotherapy target Screening computational platform we have discovered new TCRs that collectively recognize multiple small cell cancer-enhanced splicing derived epitopes. We thereby provide a general approach for expanding targeted immunotherapies. Illustrative embodiments of the present invention include methods and materials for making and using modified CD 8⁺ T cells comprising nucleic acids encoding αβ T cell receptor polypeptides selected to target cancer-enhanced splicing derived epitopes. Embodiments of the invention include, for example, a polynucleotide disposed in a vector, wherein the polynucleotide encodes a Vα T cell receptor polypeptide and a Vβ T cell receptor polypeptide disclosed herein. In typical embodiments, when a Vα/Vβ T cell receptor comprising the Vα T cell receptor polypeptide and/or the Vβ T cell receptor polypeptide is expressed in a CD 8⁺ T cell, the heterologous Vα/Vβ T cell receptor expressed on the surface of the CD 8⁺ T cell recognizes a small cell cancer-enhanced splicing peptide antigen associated with human leukocyte antigen (e.g. HLA-A*02:01). In illustrative embodiments of the invention, the peptide antigen associated with human leukocyte antigen comprises an amino acid sequence: SLAIGGVTEA (SEQ ID NO: 1); LLLGIAKLLKV (SEQ ID NO: 2); LLAEQPDQV (SEQ ID NO: 3); STMYYLWML (SEQ ID NO: 4); FLSELEPPA (SEQ ID NO: 6); FDLAYGSVITV (SEQ ID NO: 7); or SLDGTTTKA (SEQ ID NO: 8). In the illustrative working embodiments of the TCR embodiments of the invention disclosed herein, the heterologous T cell receptor comprises a Vα/Vβ T cell receptor comprising at least a 95% sequence identity to a polypeptide sequence of SEQ ID NO: 11, SEQ ID NO: 12, SEQ ID NO: 15, SEQ ID NO: 16, SEQ ID NO: 19, SEQ ID NO: 20, SEQ ID NO: 23, SEQ ID NO: 24, SEQ ID NO: 27, SEQ ID NO: 28, SEQ ID NO: 31, SEQ ID NO: 32, SEQ ID NO: 35, SEQ ID NO: 36, SEQ ID NO: 39, SEQ ID NO: 40, SEQ ID NO: 43, SEQ ID NO: 44, SEQ ID NO: 47, SEQ ID NO: 48, SEQ ID NO: 52, SEQ ID NO: 53, SEQ ID NO: 56, or SEQ ID NO: 57.

Embodiments of the invention include methods of killing cancer cells that express a small cell cancer-enhanced splicing peptide antigen, the method comprising combining the cancer cells with CD8⁺ T cells transduced with a heterologous TCR selected to recognize a small cell cancer-enhanced splicing peptide antigen associated with human leukocyte antigen under conditions that allow the heterologous TCR to be expressed on the surface of the CD8⁺ T cell and recognize small cell cancer-enhanced splicing peptides associated with a human leukocyte antigens expressed on the surface of cells of the cancer, so that the cancer cells are recognized and killed. In certain embodiments, the methods are performed in vivo on a patient infused with the CD8⁺ T cells. In certain embodiments of the invention, the patient has been diagnosed with a small cell prostate cancer or small cell lung cancer.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1. IRIS: A big-data informed platform for discovering AS-derived cancer immunotherapy targets. Workflow for IRIS, integrating computational modules, large-scale AS reference panels, and dedicated statistical testing programs. IRIS has three main modules: a, RNA-seq data processing, b, in silico screening, and c, TCR/CAR-T target prediction. On the left, a stepwise flowchart illustrates individual key components and analytical steps. d, shows the large-scale AS reference database, named IRIS DB, and visualization of screening approaches. AS, alternative splicing; CAR-T, chimeric antigen receptor T-cell; TCR, T-cell receptor.

FIG. 2. Proteo-transcriptomics analysis of HLA presentation of AS-derived peptides in normal and tumor cell lines. a, Proteo-transcriptomics workflow adopted by IRIS for discovering splice-junction peptides in MS datasets. IRIS accepts MS data inputs (right), such as whole-cell proteomics, surfaceomics, or immunopeptidomics (HLA peptidomics) data. Aided by RNA-seq inputs (left), an RNA-seq-based custom proteome library is constructed and searched using MSGF+. b, Summary of HLA presentation of AS-derived epitopes in JeKo-1 (lymphoma) and B-LCL (normal) cell lines. Peptide-spectrum matches (‘PSMs’) and ‘Unique peptides’ are provided by MSGF+ with a target-decoy FDR of 5%. ‘Predicted AS epitopes’ are generated by the IRIS prediction module, which utilizes IEDB predictors. ‘MS-validated AS epitopes’ are defined as AS epitopes that are predicted by IRIS and detected in the immunopeptidomics data. c, Percentage of IRIS-predicted AS-derived epitopes among all MS-detected epitopes for all three cell lines. Graph shows the percentage of all MS-detected epitopes that are IRIS-predicted, AS-derived peptides (y-axis) as a function of the MSGF+ target-decoy FDR (x-axis). d, Preferential detection of high-affinity AS-derived peptides in immunopeptidomics data. Graph shows the number of AS-derived peptides detected in JeKo-1 immunopeptidomics data (y-axis) as a function of the MSGF+ target-decoy FDR (x-axis). Peptides with high (IC₅₀<500 nM; orange) and low (IC₅₀≥500 nM; grey) predicted HLA binding affinities are shown. e, Heatmap depiction of the distribution of AS-derived peptides detected from JeKo-1 immunopeptidomics data as a function of predicted HLA binding affinity and transcript expression level. AS-derived peptides are binned by the corresponding transcript expression levels and IEDB-predicted binding affinity scores. Heatmap is colored from red (high) to yellow (90^{th} percentile) to blue (low), reflecting the proportion of IRIS-predicted, AS-derived epitopes that are MS-detected in each bin.

FIG. 3. IRIS identifies AS-derived targets for NEPC. a, Stepwise results of IRIS for identifying AS-derived cancer immunotherapy targets from 23 NEPC samples. Identified skipped-exon (SE) events from the IRIS data processing module were screened against a set of normal tissues from the reference panel to identify tumor-associated events and corresponding TCR targets. b, Heatmap of splicing profiles of 2,939 NEPC-associated SE events across NEPC and the selected normal tissue panel. c, Summary of identifying tumor-specific targets for NEPC. ‘Events w/Specific SJs’ are AS events that contain tumor-specific SJ(s) identified from the SJ count (SJC)-based ‘tumor-specificity screen’ and support only one form of the splicing event (e.g., either skipping form SJ or inclusion form SJs only). d, Bar plots of NEPC-associated and NEPC-specific events by event type. Events are grouped by microexon status along with inclusion (left) or skipping (right) status. e, Heatmap of gene expression profiles of 220 splicing factors across NEPC and the normal tissue panel. f, Violin plot of log-transformed gene expression levels of serine/arginine repetitive matrix protein 4 (SRRM4) across NEPC, metastatic castration-resistant prostate cancer (CRPC), primary prostate adenocarcinoma (PRAD) and the above-selected 11 normal tissues from the normal tissue panel. The two-sided Mann-Whitney U test was conducted to compare the median gene expression between NEPC samples and every other group. Red asterisks represent p-values (*: p<=0.05, **: p<=0.01, ***: p<=0.001, ****: p<=0.0001). Groups are colored by tumor (red) and normal (blue) tissue.

FIG. 4. Evaluation and visualization of IRIS-predicted targets for NEPC. a, The target evaluation process for NEPC. The three-dimensional scatterplot depicts the integrative evaluation process using results generated by IRIS. In the plot, targets are ranked based on important qualities, including their tumor association as indicated by the number of normal tissues with significant differential AS from the tumor tissues as detected in the tumor-association screens (in different colors), FC of the tumor-enriched isoform, and the gene expression level. Targets can be tailored by additional features as listed below the scatterplot. Features used in the plot are in bold font. b, Representative examples of IRIS TCR targets are visualized by IRIS in paired violin and bar plots. Each row shows paired plots for one IRIS-identified AS event. Violin plots depict the distribution of exon inclusion levels (PSI values) for each group across NEPC and the normal tissue panel. Bar plots illustrate the fraction of samples expressing the tumor SJ(s) in each group across the same data sets. If the skipping form is the tumor isoform, the bar plot displays the skipping SJ as one bar. Otherwise, two bars are displayed, representing the two inclusion SJs (5′ inclusion SJ on the left).

FIG. 5. Isolation and validation of TCRs from healthy donor PBMCs targeting IRIS-predicted AS-derived epitopes. a, IRIS-peptide priming using two APC systems: (1) differentiation of cDC1-like from CD34+ hematopoietic stem cells (HSCs) co-cultured with autologous T cells and IRIS-derived peptide pools (2) autologous T cell priming using existing APCs within PBMCs. b, Example of reactive T cell populations primed with a DMSO negative control, IRIS peptide pool, or PMA/Ionomycin using the CLInt-seq TNFα/IFNγ intracellular marker strategy. c, Example of reactive T cell populations primed with a DMSO negative control, IRIS peptide pool, or PMA/Ionomycin by CD137 surface marker staining strategy. d, Overview of cloning strategy for TCRα/β chains in the pMAX system for Jurkat-NFAT-GFP screening. e, Overview of the Jurkat-NFAT-GFP reporter system. f, IFNγ ELISA of JPTCR_238 when co-cultured with K562-A2-GFP single cell clones transduced to express a full-length or truncated AS isoform. Error bars are for standard deviation (n=3). g, Cytotoxicity analysis by live cell imagining of a K562-A2-GFP single cell clone transduced with a full-length isoform of an IRIS-predicted peptide.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention. Many of the techniques and procedures described or referenced herein are well understood and commonly employed by those skilled in the art. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

As described herein, the present invention provides methods and materials for discovering cancer-enhanced splicing derived epitopes and then making and using materials based upon these discoveries including modified T cells comprising nucleic acids encoding chimeric T cell receptor polypeptides that recognize such epitopes bound to a HLA. As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (α) and beta (β) chain, although in some cells the TCR consists of gamma and delta chains. TCRs may exist in alpha/beta and gamma/delta forms, which are structurally similar but have distinct anatomical locations and functions. Each chain is composed of two extracellular domains, a variable and constant domain. Embodiments of the invention include a number of different TCR alpha/beta nucleic acids and their encoded polypeptides.

Embodiments of the invention include compositions of matter comprising one or more vectors comprising the TCR polynucleotides disclosed herein. A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Typically, the vector is an expression vector. The term “expression” as used herein is defined as the transcription and/or translation of a particular nucleotide sequence driven by its promoter. In this context, the term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

Embodiments of the invention include, for example, a polynucleotide encoding a TCR disposed in an expression vector. Typically, a composition of the invention comprises one or more Vα/Vβ polynucleotides, for example a polynucleotide encoding a TCR Vα polypeptide in combination with a polynucleotide encoding a TCR Vβ polypeptide such that a Vα/Vβ TCR can be expressed on the surface of a mammalian cell (e.g. a CD8⁺ T cell) transduced with the vector(s), wherein the Vα/Vβ TCR recognizes a cancer-enhanced splicing derived epitope (e.g. SLAIGGVTEA (SEQ ID NO: 1); LLLGIAKLLKV (SEQ ID NO: 2); LLAEQPDQV (SEQ ID NO: 3); STMYYLWML (SEQ ID NO: 4); FLSELEPPA (SEQ ID NO: 6); FDLAYGSVITV (SEQ ID NO: 7); or SLDGTTTKA (SEQ ID NO: 8) associated with a HLA (e.g. HLA-A*02:01). The term “transduced” or “transfected” or “transformed” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

In certain compositions of the invention, a polynucleotide of the invention encodes a segment of at least 50 or at least 100 amino acids having an at least 95%, 96%, 97%, 98% or 99% sequence identity to amino acids of SEQ ID NO: 11, SEQ ID NO: 12, SEQ ID NO: 15, SEQ ID NO: 16, SEQ ID NO: 19, SEQ ID NO: 20, SEQ ID NO: 23, SEQ ID NO: 24, SEQ ID NO: 27, SEQ ID NO: 28, SEQ ID NO: 31, SEQ ID NO: 32, SEQ ID NO: 35, SEQ ID NO: 36, SEQ ID NO: 39, SEQ ID NO: 40, SEQ ID NO: 43, SEQ ID NO: 44, SEQ ID NO: 47, SEQ ID NO: 48, SEQ ID NO: 52, SEQ ID NO: 53, SEQ ID NO: 56, or SEQ ID NO: 57. In some embodiments of the invention, the T cell receptor (TCR) alpha chain polypeptide and/or the TCR beta chain polypeptide encoded by the polynucleotide comprises an amino acid substitution mutation of the wild type TCR amino acid sequence that is selected to optimize its interaction with its cognate ligand (see, e.g. Sibener et al., Cell 174, 672-687, Jul. 26, 2018; and Zhao et al., Science 376, 155 (2022), the contents of which are incorporated herein by reference). In illustrative examples of such mutants, a polynucleotide encodes a segment of at least 5, 10, 25, 50 or 100 amino acids of SEQ ID NO: 11, SEQ ID NO: 12, SEQ ID NO: 15, SEQ ID NO: 16, SEQ ID NO: 19, SEQ ID NO: 20, SEQ ID NO: 23, SEQ ID NO: 24, SEQ ID NO: 27, SEQ ID NO: 28, SEQ ID NO: 31, SEQ ID NO: 32, SEQ ID NO: 35, SEQ ID NO: 36, SEQ ID NO: 39, SEQ ID NO: 40, SEQ ID NO: 43, SEQ ID NO: 44, SEQ ID NO: 47, SEQ ID NO: 48, SEQ ID NO: 52, SEQ ID NO: 53, SEQ ID NO: 56, or SEQ ID NO: 57.

In certain embodiments of the invention, the T cell receptor comprises at least one polypeptide sequence having at least a 95%-99% sequence identity to SEQ ID NO: 11, SEQ ID NO: 12, SEQ ID NO: 15, SEQ ID NO: 16, SEQ ID NO: 19, SEQ ID NO: 20, SEQ ID NO: 23, SEQ ID NO: 24, SEQ ID NO: 27, SEQ ID NO: 28, SEQ ID NO: 31, SEQ ID NO: 32, SEQ ID NO: 35, SEQ ID NO: 36, SEQ ID NO: 39, SEQ ID NO: 40, SEQ ID NO: 43, SEQ ID NO: 44, SEQ ID NO: 47, SEQ ID NO: 48, SEQ ID NO: 52, SEQ ID NO: 53, SEQ ID NO: 56, or SEQ ID NO: 57. Optionally for example, the T cell receptor comprises at least one polypeptide sequence comprising 1, 2 or 3 amino acid substitution mutations in SEQ ID NO: 11, SEQ ID NO: 12, SEQ ID NO: 15, SEQ ID NO: 16, SEQ ID NO: 19, SEQ ID NO: 20, SEQ ID NO: 23, SEQ ID NO: 24, SEQ ID NO: 27, SEQ ID NO: 28, SEQ ID NO: 31, SEQ ID NO: 32, SEQ ID NO: 35, SEQ ID NO: 36, SEQ ID NO: 39, SEQ ID NO: 40, SEQ ID NO: 43, SEQ ID NO: 44, SEQ ID NO: 47, SEQ ID NO: 48, SEQ ID NO: 52, SEQ ID NO: 53, SEQ ID NO: 56, or SEQ ID NO: 57.

In certain embodiments of the invention, the vector is a Sendai viral vector, an adenoviral vector, an adeno-associated virus vector, a retroviral vector, or a lentiviral vector. In typical embodiments, the vector comprises a polynucleotide encoding a Vα polypeptide in combination with a polynucleotide encoding a Vβ polypeptide disposed in the vector so that a Vα/Vβ T cell receptor (TCR) is expressed on the surface of a CD 8⁺ T cell.

Embodiments of the invention also include composition of matter comprising a host cell transduced with a vector disclosed herein. In illustrative embodiments of the invention, the host cell is a human CD 8⁺ T cell. Typically, the composition is a pharmaceutical composition comprising one more pharmaceutically acceptable excipients selected from the group consisting of buffering agents, antimicrobial agents, tonicity adjusting agents, wetting agents, detergents and pH adjusting agents. In certain embodiments of the invention, the CD8⁺ T cell is obtained from an individual diagnosed with a cancer that expresses a small cell cancer-enhanced splicing peptide antigen; and the CD8⁺ T cell is transduced with a vector (e.g. a retroviral vector) comprising a polynucleotide encoding a TCR Vα polypeptide in combination with a polynucleotide encoding a TCR Vβ polypeptide such that a heterologous TCR is expressed on a surface of the CD8⁺ T cell, wherein the heterologous TCR recognizes a small cell cancer-enhanced splicing peptide antigen associated with a human leukocyte antigen expressed on the surface of cells of the cancer.

Embodiments of the invention also include methods of killing cancer cells that express a small cell cancer-enhanced splicing peptide antigen, the method comprising combining the cancer cells with CD8⁺ T cells transduced with a vector disclosed herein under conditions that allow a heterologous TCR to be expressed on the surface of the CD8⁺ T cell and recognize small cell cancer-enhanced splicing peptides associated with a human leukocyte antigens expressed on the surface of cells of the cancer, so that the cancer cells are recognized and killed. In certain embodiments of the invention, the method is performed in vivo on a patient infused with the CD8⁺ T cells. In certain embodiments of the invention, the cancer cells form small cell tumors. In some embodiments of the invention, the cancer cells are small cell prostate cancer cells or small cell lung cancer cells. In some embodiments, the method comprises administering a first modified CD8⁺ T cell that targets a small cell cancer-enhanced splicing peptide antigen associated with a first human leukocyte antigen human leukocyte antigen in combination with a second CD8⁺ T cell that targets a small cell cancer-enhanced splicing peptide antigen associated with second human leukocyte antigen.

Embodiments of the invention also include use of a polynucleotide or CD8⁺ T cell disclosed herein for the manufacture of a medicament for the treatment of a cancer (e.g., a small cell prostate cancer or a small cell lung cancer). Typically, the polynucleotide comprises at least one polynucleotide encoding a polypeptide sequence having at least a 95% sequence identity to SEQ ID NO: 11, SEQ ID NO: 12, SEQ ID NO: 15, SEQ ID NO: 16, SEQ ID NO: 19, SEQ ID NO: 20, SEQ ID NO: 23, SEQ ID NO: 24, SEQ ID NO: 27, SEQ ID NO: 28, SEQ ID NO: 31, SEQ ID NO: 32, SEQ ID NO: 35, SEQ ID NO: 36, SEQ ID NO: 39, SEQ ID NO: 40, SEQ ID NO: 43, SEQ ID NO: 44, SEQ ID NO: 47, SEQ ID NO: 48, SEQ ID NO: 52, SEQ ID NO: 53, SEQ ID NO: 56, or SEQ ID NO: 57.

In another aspect, the invention includes a method for generating a modified T cell comprising introducing one or more nucleic acids (e.g., nucleic acids disposed within a lentiviral vector) encoding a TCR disclosed herein into a T cell (e.g. a CD8⁺ T cell obtained from an individual diagnosed with a cancer that expresses a small cell cancer-enhanced splicing peptide antigen). The present invention also includes modified T cells with downregulated or knocked out gene expression (e.g., a modified T cell having a knocked out endogenous T cell receptor and an exogenous/introduced T cell receptor that recognizes a small cell cancer-enhanced splicing peptide antigen associated with a HLA). The term “knockdown” as used herein refers to a decrease in gene expression of one or more genes. The term “knockout” as used herein refers to the ablation of gene expression of one or more genes.

The modified T cells described herein may be included in a composition for use in a therapeutic regimen. The composition may include a pharmaceutical composition and further include a pharmaceutically acceptable carrier. A therapeutically effective amount of the pharmaceutical composition comprising the modified T cells may be administered. Pharmaceutical compositions of the present invention may comprise the modified T cell as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Adoptive immunotherapy with T cells harboring antigen-specific TCRs have therapeutic potential in the treatment of cancers. Gene-engineering of CD 8⁺ T cells with a specific TCR has the advantage of redirecting the T cell to a selected antigen such as an small cell cancer-enhanced splicing peptide antigen. In this context, in one aspect, the invention includes methods for stimulating a T cell-mediated immune response to a target cell or tissue in a subject comprising administering to a subject an effective amount of a modified CD 8⁺ T cell. In this embodiment, the CD8⁺ T cell is modified as described elsewhere herein. Embodiments of the invention also include administering multiple modified CD 8⁺ T cells that target multiple small cell cancer-enhanced splicing peptide antigen epitopes. For example, embodiments of the invention include administering at least two different modified CD8⁺ T cells, for example a first modified CD8⁺ T cell that targets a small cell cancer-enhanced splicing peptide associated with a first human leukocyte antigen human leukocyte antigen in combination with a second CD8⁺ T cells that targets a small cell cancer-enhanced splicing peptide antigen associated with second human leukocyte antigen.

Embodiments of the invention encompass methods of treating a disease or condition characterized by the expression of small cell cancer-enhanced splicing peptide antigen. The treatment methodology comprises comprising administering an effective amount of a pharmaceutical composition comprising the modified T cell described herein to a subject in need thereof. The term “subject” is intended to include living organisms in which an immune response can be elicited (e.g., mammals). A “subject” or “patient”, as used therein, may be a human or non-human mammal. Non-human mammals include, for example, livestock and pets, such as ovine, bovine, porcine, canine, feline and murine mammals. Preferably, the subject is human. In typical embodiments of the invention, the human has a cancer expressing a polypeptide that functions as a small cell cancer-enhanced splicing peptide antigen. In some embodiments of the invention, the cells of the cancer form solid tumors.

A related embodiment of the invention includes a method for prophylaxis and/or therapy of an individual diagnosed with, suspected of having or at risk for developing or recurrence of a cancer, wherein the cancer comprises cancer cells which express a small cell cancer-enhanced splicing peptide antigen. This approach comprises administering to the individual modified human T cells comprising a recombinant polynucleotide encoding a TCR, wherein the T cells are capable of direct recognition of the cancer cells expressing the small cell cancer-enhanced splicing peptide antigen, and wherein the direct recognition of the cancer cells comprises HLA class II-restricted binding of the TCR to the small cell cancer-enhanced splicing peptide antigen expressed by the cancer cells.

With respect to use of the engineered CD8⁺ T cells of the present invention, the method generally comprises administering an effective amount (e.g. by intravenous or intraperitoneal injections) of a composition comprising the CD8⁺ T cells to an individual in need thereof. An appropriate pharmaceutical composition may be adapted for administration by any appropriate route, such as parenteral (including subcutaneous, intramuscular, or intravenous), enteral (including oral or rectal), inhalation or intranasal routes. Such compositions may be prepared by any method known in the art of pharmacy, for example by mixing the active ingredient with the carrier(s) or excipient(s) under sterile conditions.

In another aspect, the invention includes use of a polynucleotide or a modified CD8⁺ T cell described herein in the manufacture of a medicament for the treatment of a disease or condition characterized by the expression of a small cell cancer-enhanced splicing peptide antigen, in a subject in need thereof. In illustrative embodiments of the invention, the disease is a cancer expressing small cell cancer-enhanced splicing peptide antigen, for example a small cell lung cancer.

The technology in this area is fairly developed and a number of methods and materials know in this art can be adapted for use with the invention disclosed herein. Such methods and materials are disclosed, for example in U.S. Patent Publication Nos. 20190247432, 20190119350, 20190002523, 20190002522, 20180371050, 20180057560, 20170029483, 20160024174, and 20150141347, the contents of which are incorporated by reference.

Further aspects and embodiments of the invention are provided in the examples below.

### EXAMPLES

### Example 1: Cancer Immunotherapy Targets Arising from Pre-mRNA Alternative Splicing

Cancer immunotherapy has gained remarkable success in the past decade. Checkpoint inhibitors, like neutralizing PD-1 and CTLA-4 antibodies, are thought to be clinically effective by reactivating tumor-specific T cells (1). In contrast, adoptive cell therapies use genetically modified T-cell receptors (TCR) and chimeric antigen receptor T-cells (CAR-T) to target antigens expressed in cancer cells (2). The insight that cancer cells express specific T-cell-reactive antigens has galvanized antigen discovery efforts in recent years (3-6). Nevertheless, the discovery of tumor antigens (TAs) remains a major challenge (7, 8). Although somatic mutation-derived TAs have been successfully targeted by cancer therapies (9-12), this approach remains largely ineffective for tumors with low or moderate mutation load (3).

Post-transcriptional RNA processing is an essential layer of eukaryotic gene expression, and its dysregulation has a major impact on the cancer cell proteome (13-15). Various types of RNA-level dysregulation can generate aberrant proteins and immunogenic peptides in cancer cells (16-20). In a pan-cancer analysis, Kahles et al. found that tumors harbor up to 30% more alternative splicing (AS) events than normal tissues, and some of the resulting peptides are predicted to be presented by HLA molecules (16). In another study, experimental evidence of HLA class I (HLA-I) presentation of peptides derived from intron retention, a specific type of AS, was reported based on mass spectrometry (MS) proteomics data (17). These findings have inspired a growing interest in AS as a rich source of potential immunotherapy targets (14, 21).

Currently, there are limited computational tools for discovering AS-derived TAs. Two recently published tools, ASNEO (22) and NeoSplice (23), sought to discover AS-derived TCR targets for cancer immunotherapy. Both tools use RNA-seq data of tumor tissues as well as selected normal tissues to identify putative tumor-specific splice junctions, followed by HLA binding prediction. However, they lack the computational infrastructure to leverage large cohorts of tumor and normal transcriptomes in public repositories to comprehensively determine the tumor association and specificity of predicted targets. Importantly, neither studies experimentally tested the immunogenicity of predicted targets or their ability to activate functional T-cell responses.

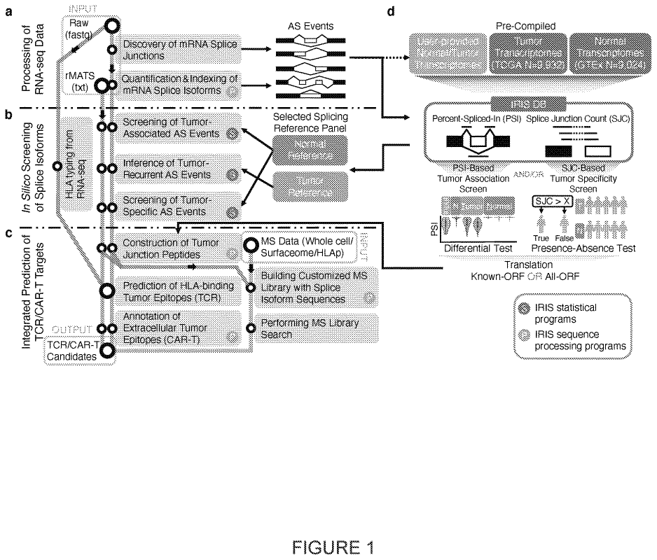

We have developed an in silico platform to discover and prioritize AS-derived immunotherapy targets of varying degrees of tumor specificity, by utilizing an ‘AS reference’ that represents splicing profiles of tens of thousands of tumor and normal transcriptomes generated by large-scale consortia (e.g., GTEx, TCGA) (24, 25). Our platform, Isoform peptides from RNA splicing for Immunotherapy target Screening (IRIS), enables a big-data informed discovery of AS-derived TCR and CAR-T targets through a streamlined framework (FIG. 1a). IRIS is powered by the new generation of our widely used rMATS software (rMATS-turbo) (26) for AS analysis of RNA-seq data, with a substantial improvement in speed and computational efficiency enabling ultra-fast analyses of AS events across massive RNA-seq datasets. We initially tested the utility of IRIS through a proof-of-concept analysis using immunopeptidomics data of human cell lines. We then applied IRIS to RNA-seq data of neuroendocrine prostate cancer, a metastatic and highly lethal prostate cancer with no effective long-term treatments or targeted therapies (27). To validate the immunogenicity and T-cell recognition of IRIS-predicted TCR targets, we performed an in vitro T-cell priming assay in combination with single-cell TCR sequencing, followed by reconstitution and functional characterization of TCRs transduced into human peripheral blood mononuclear cells (PBMCs). Collectively, our study illustrates the contribution of AS to the TA repertoire of cancer cells and demonstrates the utility of IRIS for discovering novel TAs and expanding targeted cancer immunotherapies.

### Results

### Overall Design of the IRIS Computational Framework

To identify AS-derived immunotherapy targets, IRIS incorporates three main modules: processing of RNA-seq data, in silico screening for tumor-associated or tumor-specific AS events, and integrated prediction and prioritization of TCR and CAR-T targets (FIG. 1). Briefly, IRIS first discovers and quantifies various types of AS events from user-provided RNA-seq data of a given tumor type (FIG. 1a). Then, AS events are fed into an in silico screening module to identify tumor-associated or tumor-specific events, based on a comparison against large-scale reference RNA-seq resources of tumor and normal tissues (FIG. 1b). Lastly, IRIS performs TCR and CAR-T target prediction for the identified AS events (FIG. 1c).

In IRIS's RNA-seq data processing module, user-provided RNA-seq data of a given tumor type is analyzed by the rMATS-turbo software to comprehensively discover and quantify AS events corresponding to major types of AS patterns (FIG. 1a). The rMATS/rMATS-turbo software was developed by our group for AS analysis of RNA-seq data and has been widely used by the research community since 2014 (26, 28). Compared to the original rMATS software (28), rMATS-turbo incorporates a refactored computational workflow with substantially improved data processing speed and efficiency, allowing it to scale up to massive RNA-seq datasets with tens of thousands of samples (26). Powered by the speed and efficiency of rMATS-turbo, we uniformly processed 18,956 RNA-seq samples in public data repositories generated by large-scale consortia (TCGA, GTEx), representing 33 tumor types and 51 normal tissue types from 30 histological sites. Results of this analysis were organized into the IRIS Alternative Splicing Database (IRIS DB), which contains ratio-based [percent-spliced-in (PSI)] (29) and count-based [splice junction (SJ) read count] quantification for all major types of AS events detected in TCGA and GTEx (FIG. 1d). The IRIS DB is indexed, allowing for the efficient querying of AS events in large-scale tumor and normal transcriptomes from diverse tumor types and tissue origins.

IRIS's in silico screening module provides three distinct screening tests to identify targets of varying degrees of tumor association and specificity (FIG. 1b). Specifically, IRIS compares AS events from user-provided RNA-seq data of a given tumor type to a reference panel of user-specified tumor and normal tissues selected from the IRIS DB. The default ‘tumor-association screen’ uses the PSI metric to identify tumor-associated AS events, via a differential AS (PSI value) analysis between tumor and normal tissues based on various user-defined criteria, such as p-value and change of PSI value (delta PSI), as well as fold-change (FC) of tumor-enriched isoform. Moreover, to identify AS events with ‘neoantigen-like’ tumor specificity, defined as AS-derived SJs that are exclusively expressed in tumor tissues, IRIS performs a highly stringent ‘tumor-specificity screen’ by testing and comparing the presence-absence of a given SJ between tumor and normal tissues. Specifically, for each sample group (e.g. user-provided tumor RNA-seq samples, or a reference normal tissue type in the IRIS DB), IRIS calculates the percentage of samples expressing a given SJ of interest above a user-defined read count threshold. IRIS then performs a Fisher Exact test to identify ‘tumor-specific’ SJs that are expressed in a significantly higher percentage of tumor samples than in normal tissue samples. IRIS reports a tumor-associated AS event as tumor-specific if it contains a tumor-specific SJ identified by the tumor-specificity screen. Finally, IRIS also incorporates a ‘tumor-recurrence screen’ to compare AS events between user-provided RNA-seq samples of a given tumor type to user-selected tumor types of similar histology in the IRIS DB. This test allows IRIS to identify AS events that are recurrent (shared) among independent cohorts of the similar tumor type.

IRIS's target prediction module incorporates various prediction tools and annotation resources to identify candidate targets for immunotherapies (FIG. 1c). The module first constructs SJ peptides of identified AS events, and then predicts AS-derived targets for TCR or CAR-T therapies. The TCR target prediction first performs tumor HLA typing using RNA-seq data or accepts user-specified HLA types, then integrates multiple HLA-binding prediction algorithms for predicting TCR targets and/or peptide vaccines. Specifically, IRIS uses Immune Epitope Database (IEDB) (30) predictors to obtain the putative HLA binding affinities of candidate peptides. The IEDB ‘recommended’ mode runs multiple prediction tools to generate multiple predictions of binding affinity, which IRIS summarizes as a median IC₅₀ value. In parallel, CAR-T target prediction maps AS-derived peptides to protein extracellular domain annotations curated by UniProtKB (31). IRIS also includes an option to confirm predicted AS-derived targets using MS data via proteo-transcriptomics data integration (FIG. 1c). This option provides an orthogonal approach for target discovery and validation by integrating RNA-seq data with various types of MS data, such as whole-cell proteomics, surfaceomics, or immunopeptidomics data. Specifically, IRIS builds a custom library of AS-derived peptides and then searches MS spectra against this library, allowing proteomic validation of AS-derived targets using MS data.

### AS-Derived Peptides are Present in Cell Line Immunopeptidomes

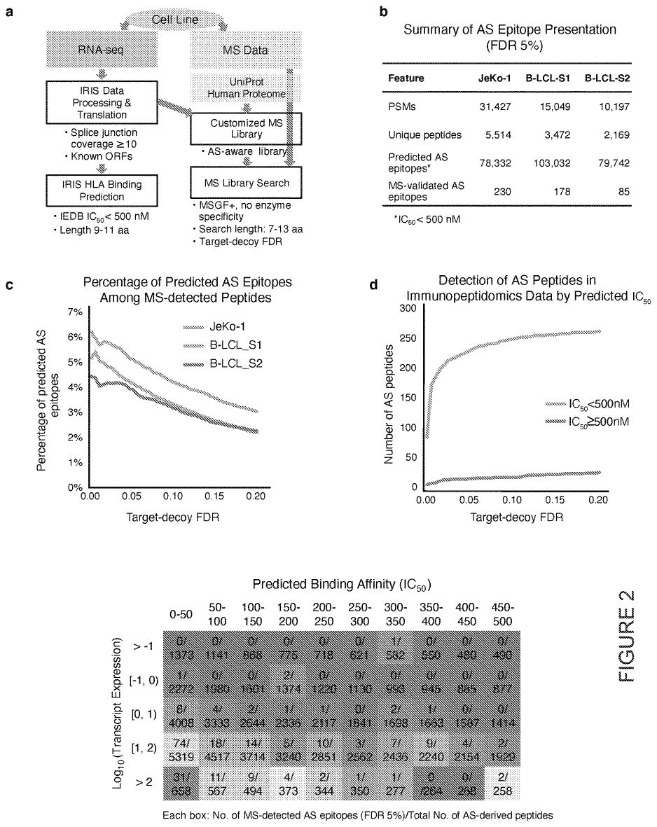

In a proof-of-concept analysis, we sought to identify AS-derived peptides that are presented by HLA molecules, i.e. AS-derived epitopes, by applying IRIS's RNA-seq data processing and target prediction modules to RNA-seq and MS-based immunopeptidomics data of multiple cell lines (FIG. 2a). Specifically, we analyzed paired RNA-seq and immunopeptidomics data of two B lymphoblastoid cell lines (B-LCL) (32) and one cancer cell line (JeKo-1 lymphoma) (33). Focusing on predicting HLA-I binding to AS-derived peptides, we found 230, 178, and 85 peptides present in the immunopeptidomics data of JeKo-1, B-LCL-S1, and B-LCL-S2, respectively, after controlling for the target-decoy false discovery rate (FDR) at 5% (FIG. 2b). Our results provide evidence that AS-derived peptides are presented by HLA-I molecules.

We assessed the concordance between AS-derived epitopes predicted by HLA binding algorithms (‘IEDB recommended’) and those detected from immunopeptidomics data. For all three cell lines, the percentage of AS-derived epitopes among all epitopes detected from immunopeptidomics data increased progressively with more stringent target-decoy FDR cutoffs (FIG. 2c). Among all epitopes detected from immunopeptidomics data, AS-derived peptides with high predicted HLA binding affinities (IC₅₀<500 nM) substantially outnumbered AS-derived peptides with low predicted HLA binding affinities (IC₅₀≥500 nM) (see FIG. 2d for data on JeKo-1). Moreover, in all three cell lines we observed an increase in the fraction of AS-derived peptides detected from immunopeptidomics data as a function of higher transcript expression levels and higher predicted HLA-binding affinities (see FIG. 2e for data on JeKo-1). For example, only 52 out of 56,254 IRIS-predicted AS-derived epitopes were detected from immunopeptidomics data when the transcript expression level was lower than 10 fragments per kilobase million (FPKM) or the predicted HLA binding affinity was weaker than IC₅₀ of 250 nM. In contrast, 178 out of 22,077 AS-derived epitopes were detected from immunopeptidomics data when the transcript expression level was higher than 10 FPKM and the predicted HLA binding affinity was stronger than IC₅₀ of 250 nM. The largest fraction of AS-derived epitopes detected from immunopeptidomics data were observed in the bottom leftmost bin of FIG. 2e; these represent AS-derived epitopes with the highest transcript expression level (>100 FPKM) and strongest predicted HLA binding affinity (IC₅₀<50 nM). Our results demonstrate that AS-derived epitopes supported by immunopeptidomics data are enriched for transcripts with high expression levels and peptides with strong predicted HLA binding affinities, consistent with the expected pattern of HLA-epitope binding (34).

### Discovery of AS-Derived Immunotherapy Targets for Neuroendocrine Prostate Cancers

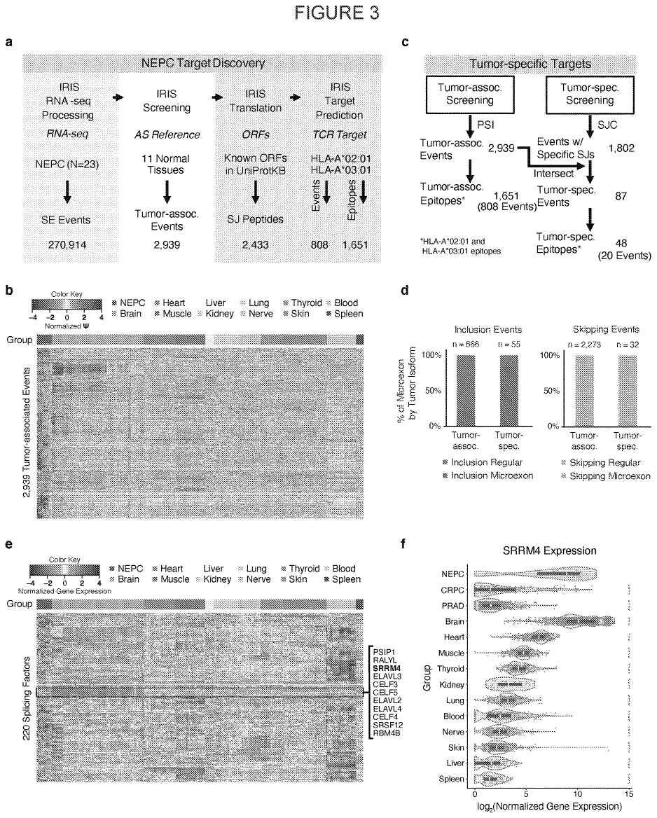

To demonstrate the utility of IRIS in discovering AS-derived immunotherapy targets in tumor specimens, we applied IRIS to a published RNA-seq dataset of 23 neuroendocrine prostate cancer (NEPC) samples (FIG. 3a). For the normal tissue panel, we selected 11 vital tissues from the IRIS DB. In total, 270,914 skipped exon (SE) events were identified and quantified in the NEPC dataset (FIG. 3a; blue panel). Using the PSI-based tumor-association screen, IRIS identified 2,939 SE events as tumor-associated (FIG. 3a; yellow panel). As illustrated in FIG. 3b, hierarchical clustering based on PSI values confirmed that NEPC-associated SE events have distinct splicing profiles in most of the normal tissue types and modestly similar splicing profiles in normal brain as compared to the splicing profiles in NEPC.

Next, for each NEPC-associated SE event, SJ(s) of the tumor-enriched isoform (i.e., the isoform that is more abundant in the tumor samples compared to the normal tissue panel) were translated into peptides, followed by TCR target prediction (FIG. 3a; purple panel). From 2,939 NEPC-associated SE events, 2,433 tumor-enriched SJs can be translated into peptide sequences using annotated open reading frames (ORFs). Of these, 1,651 epitopes from 808 NEPC-associated SE events were predicted as TCR targets for two common HLA types, HLA-A*02:01 and HLA-A*03:01. Using the same procedure, we identified 385 epitopes from 207 NEPC-associated AS events corresponding to alternative 5′ splice sites (A5SS), 3′ splice sites (A3SS), and retained introns (RI) as additional TCR targets.

IRIS also identifies tumor-associated SJ peptides located in annotated extracellular regions of cell-surface proteins. In total, 168 NEPC-associated AS events, including 119 SE events, were identified as located in extracellular regions of cell-surface proteins. Such events may represent potential CAR-T targets.

### NEPC-Specific SE Events are Enriched for Microexons

To prioritize targets with greater tumor specificity, we used IRIS to perform a more stringent ‘tumor-specificity screen’ by testing and comparing the presence-absence of a given SJ between tumor and normal tissues (FIG. 3c). This screen identified 1,802 SE events with NEPC-specific SJs. Intersecting these events with the 2,939 NEPC-associated SE events identified by the tumor-association screen yielded a prioritized set of 87 NEPC-specific SE events that could potentially produce ‘neoantigen-like’ AS-derived targets. Of the NEPC-specific peptides encoded by these events, 48 epitopes from 20 events were predicted to bind to HLA-A*02:01 or HLA-A*03:01.

We found that these 87 NEPC-specific SE events were significantly enriched for events corresponding to NEPC-specific inclusion of microexons (i.e., exons no more than 30 nucleotides in length (35)), compared to the 2,939 NEPC-associated events (FIG. 3d). Among the 55 events corresponding to NEPC-specific exon inclusion, 46 (83.6%) involved a microexon, a significant increase as compared to the 145 among 666 (21.8%) events corresponding to NEPC-associated exon inclusion involving microexons (P=7.6×10⁻²⁵) (FIG. 3d; left panel). In comparison, the percentage of events involving microexons was much lower for those corresponding to NEPC-specific or NEPC-associated exon skipping (2 out of 32 [6.3%] and 56 out of 2,273 [2.5%], respectively; FIG. 3d; right panel). To investigate whether NEPC-specific microexon inclusion is correlated with the expression of splicing factors, we examined gene expression levels of 220 splicing factors (36) across NEPC and the normal tissue panel. Hierarchical clustering of splicing factor gene expression levels revealed a cluster of 11 splicing factors with elevated expression in NEPC and normal brain compared to the rest of the normal tissue panel (FIG. 3e). Notably, serine/arginine repetitive matrix 4 (SRRM4), which was previously reported to promote neuronal-specific inclusion of microexons through an evolutionarily conserved mechanism (35), was among the 11 splicing factors overexpressed in NEPC and normal brain. Further comparison of SRRM4 gene expression levels among NEPC, metastatic castration-resistant prostate cancer (CRPC), and primary prostate adenocarcinoma (PRAD) samples revealed that overexpression of SRRM4 is unique to NEPC (FIG. 3f), a finding that is consistent with a previous report (37). Together, these observations point to SRRM4, among other splicing factors, as a likely contributor of NEPC-specific inclusion of microexons and consequently the TA repertoire of NEPC.

### Big-Data Informed Evaluation and Visualization of AS-Derived Immunotherapy Targets

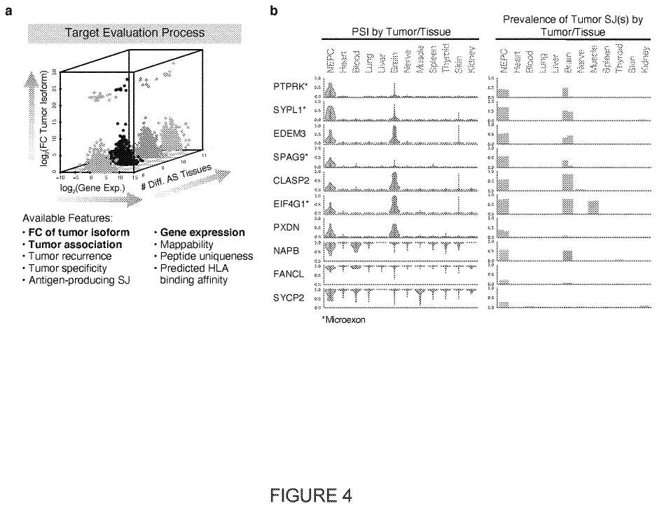

IRIS generates an integrated report that allows researchers to evaluate and visualize predicted targets based on multiple criteria (FIG. 4). The three main criteria are: degree of tumor association, FC of the tumor-enriched isoform between tumor and normal tissues, and gene expression level in tumor tissues (see FIG. 4a for visualization of these criteria for predicted tumor-associated NEPC targets). The degree of tumor association is represented as the number of normal tissue types 1) with significant differential AS and 2) the same delta PSI direction from the tumor tissues as detected in the tumor-association screen. The ‘FC of tumor-enriched isoform’ is calculated as the fold change of the proportion of the tumor-enriched isoform in tumor tissues over the average proportion of the tumor-enriched isoform in all normal tissue types of the normal tissue panel. The gene expression level is the median expression level of the AS gene in the tumor tissues. IRIS also generates additional features for predicted targets, including tumor specificity, predicted HLA binding affinity, as well as genome or protein annotations (e.g. mappability, peptide uniqueness, etc.).

Representative examples of 10 NEPC-associated or NEPC-specific (the last 2 and the first 8, respectively) TCR targets are shown in paired violin and bar plots generated by IRIS (FIG. 4b). To illustrate tumor association, violin plots show the exon inclusion level (i.e. ‘PSI’ value) of each target in NEPC and the normal tissue panel (FIG. 4b; left panel). To visualize tumor specificity, bar plots show the fraction of samples expressing the SJ(s) of the tumor-enriched isoform in NEPC and the normal tissue panel (FIG. 4b; right panel). As expected, predicted TCR targets display distinct splicing profiles in NEPC relative to most of the normal tissue types, with the occasional exception being the normal brain. For example, an SE event in protein tyrosine phosphatase receptor type K (PTPRK) is selected by both tumor-association and tumor-specificity screens, with the exon included isoform being the tumor-enriched isoform. Its tumor association is reflected by violin plots of PSI values, showing that the SE event has an average PSI value of 27% among NEPC samples as compared to almost 0% (no exon inclusion) across the normal tissue panel. The bar plots show that the two SJs of the exon included isoform are present in approximately half of NEPC samples, while absent in nearly all tissue types in the normal tissue panel except for one SJ in the normal brain. Likewise, a known microexon target of SRRM4 in eukaryotic translation initiation factor 4 gamma 1 (EIF4G1) (38) exhibits elevated exon inclusion in NEPC (and in brain) as shown by both screens (FIG. 4b). To facilitate data exploration, we developed a web-based interactive visualization interface as part of the IRIS Explorer (https://xingshiny2.research.chop.edu/shiny/IRIS/, under the ‘Browse Data’ tab) to generate plots based on tumor-association and tumor-specificity screens.

### Isolation and Characterization of TCRs Reactive to IRIS-Predicted NEPC Epitopes

From 1,651 NEPC-associated epitopes, 76 unique epitopes were selected from 216 epitopes that met additional criteria for FC of the tumor-enriched isoform and gene expression level in tumor tissues and had predicted HLA-A*02:01 affinity <500 nM (30). These epitopes were selected as candidates to study their immunogenicity and identify their cognate TCRs.

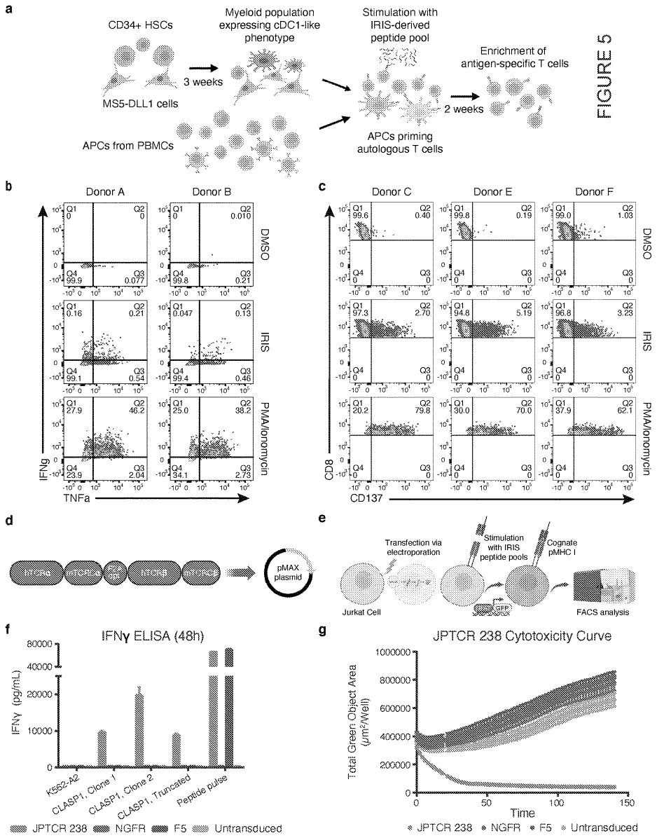

To expand and isolate cognate T cells targeting predicted epitopes, peripheral blood mononuclear cells were stimulated with exogenously added peptides using two types of antigen-presenting cell (APC) systems, including: 1) dendritic cells (DCs) differentiated from autologous CD34+ progenitor cells, and 2) existing APCs (e.g. B cells, monocytes) from PBMCs (FIG. 5a). Following 10 days of priming and expansion, reactive T cells were isolated by fluorescence-activated cell sorting (FACS) based on either a surface activation marker (CD137) or intracellular markers (IFNγ and TNFα) using a previously published CLInt-seq technique (39-41). 10X single-cell V(D)J sequencing was performed to recover paired TCR sequences. PBMCs from nine healthy individuals were screened. Five donors showed T cell responses when stimulated by IRIS-predicted epitope pool by either CLInt-seq (FIG. 5b) or CD137 (FIG. 5c).

Isolated candidate TCRs were tested in a Jurkat-NFAT-GFP reporter system for rapid functional screening and cognate peptide deconvolution. NFAT-binding motifs followed by a GFP expression sequence were introduced in Jurkat cells co-expressing CD8. Upon T cell activation, GFP expression will be induced by transcription factor NFAT (42). TCRα/β pairs isolated from sequencing were synthesized and reconstructed into a single fragment by F2Aopt linker in the pMAX plasmid to ensure equal copies of both alpha and beta chains (FIG. 5d). Plasmids were then transfected into Jurkat-NFAT-GFP cells via electroporation. For higher throughput, we applied a pooling strategy to deconvolute reactive pools to a single peptide (FIG. 5e). A total of 22 TCRs derived from the five healthy donors recognized the peptide pool. Of these, seven TCRs reacted to a single IRIS-predicted epitope in Jurkat-NFAT-GFP cells.

TCRs that showed a response in the Jurkat-NFAT-GFP screening were then selected for engineering into healthy donor PBMCs via retroviral transduction to confirm functional reactivity and cytotoxicity. When expressed in PBMCs, the seven TCRs recognized four exogenously added IRIS-predicted epitopes, as measured by the production of IFNγ. One TCR (JPTCR_47) showed similar reactivity toward its target as compared to the clinically tested F5 TCR, as measured by peptide serial-dilution assays (43).

To test if the isolated TCRs could recognize processed epitopes on HLA-A*02:01, truncated isoforms for five TCR-peptide pairs were introduced into target K562 cells that co-express A*02:01 (K562-A2). IFNγ ELISA results confirmed the reactivity JPTCR_238 in PBMCs when cocultured with both truncated and full-length cytoplasmic linker associated protein 1 (CLASP1) isoforms containing the splicing event of interest (FIG. 5f). Cytotoxicity results measured by Incucyte live-cell analysis showed recognition and killing of target cells by JPTCR_238 (FIG. 5g). Taken together, our data provide experimental evidence that antigen-reactive TCRs can target IRIS-predicted AS-derived epitopes with high potency and specificity.

We introduce IRIS, a computational framework that leverages large-scale RNA-seq data for the discovery of AS-derived TAs. We demonstrated the utility of IRIS with a proof-of-concept analysis using paired RNA-seq and immunopeptidomics data of three human cell lines. We performed an in-depth analysis of a metastatic and highly lethal prostate cancer, NEPC, to evaluate the ability of IRIS to discover AS-derived immunotherapy targets in tumor specimens. The NEPC-specific AS events we identified were highly enriched for microexons, pointing to a distinct program of splicing dysregulation in this aggressive disease (37). By employing in vitro T cell priming and subsequent single-cell TCRα/β sequencing based on T cell activation markers, we established that IRIS-predicted NEPC epitopes could be recognized by T cells. These data provide large-scale experimental evidence for antigen-reactive TCR efficacy against AS-derived epitopes.

IRIS represents a systematic and generalizable strategy for exploiting AS as a novel source of cancer immunotherapy targets. By performing multiple types of in silico screening tests against a large-scale reference database of AS profiles of tumor and normal tissues (IRIS DB), IRIS can identify and prioritize AS-derived targets with varying degrees of tumor association and specificity. Importantly, to prioritize tumor-specific targets, IRIS incorporates a SJ count-based ‘tumor-specificity screen’ to test the presence-absence of any given SJ in tumor and normal tissue samples, allowing detection of AS-derived targets with neoantigen-like tumor specificity. Additionally, by examining RNA-seq data from the same cancer type through the “tumor-recurrence” test, IRIS can discover TAs shared among patients from different cohorts. Collectively, IRIS's ability to perform comprehensive screening tests along with its associated data resource (‘IRIS DB’) provides a significant advantage over existing target discovery pipelines (16, 17, 22, 23) and facilitates selection of AS-derived targets with low off-tumor toxicity and broad clinical applicability.

We assessed and validated IRIS-predicted epitopes via independent approaches. We initially performed a proof-of-concept analysis integrating RNA-seq and immunopeptidomics data, and confirmed the presence of IRIS-predicted epitopes in the HLA-I immunopeptidome of multiple human cell lines (FIG. 2). As expected, predicted AS-derived epitopes from transcripts with higher expression levels and corresponding to peptides with stronger predicted HLA-binding affinities are more likely to be detected in immunopeptidomics data (FIG. 2e). We then applied IRIS for TCR target discovery for NEPC, a highly lethal prostate cancer with no effective long-term treatments or targeted therapies. IRIS identified 2,939 tumor-associated SE events, among which 87 were identified as tumor-specific. Of note, tumor-specific SE events were significantly enriched for NEPC-specific inclusion of microexons (FIG. 3d), which are known to be upregulated in neuronal cell lineages and may underlie the neuroendocrine transformation of prostate cancer cells in NEPC (44). We noted that enhanced expressions of splicing factor SRRM4 in NEPC correlated with these results. We experimentally isolated seven unique TCRs specifically recognizing four unique IRIS-predicted epitopes. We tested one TCR that showed efficient killing of target cells expressing the full-length target protein. Our work has demonstrated that AS-derived epitopes predicted by IRIS can be processed and presented on HLA-I and recognized by a cognate TCR discovered from healthy donor PBMCs. Further application of the workflow shown here should eventually result in significant new targets and therapeutic TCRs for many types of cancer.

The current IRIS platform has several limitations. IRIS uses short-read RNA-seq data for AS analysis. Although short-read RNA-seq has been the standard technology for transcriptome analysis, it has an inherent limitation for inferring full-length transcript isoforms and their corresponding protein products (45). Since short-read RNA-seq only examines fragments of full-length transcripts, protein products that correspond to the identified AS events often cannot be reliably inferred, particularly for events involving complex AS patterns or novel unannotated SJs. Currently, target discovery in IRIS is limited to peptides encoded by SJs corresponding to basic types of binary AS patterns (FIG. 1); thus, a considerable number of potential epitopes including those derived from complex AS events, are not considered. The emerging long-read RNA-seq technology, which is ideally suited for analyzing full-length transcript and protein isoforms, may overcome this limitation of short-read RNA-seq and enable a more comprehensive approach for TA discovery (14, 46, 47). Additionally, the current IRIS platform and its associated IRIS DB are based on bulk RNA-seq data and lack single-cell or spatial resolution. In the future, isoform-resolved single-cell or spatial RNA-seq datasets may enhance the resolution of transcriptome references in IRIS, by providing cell type-specific or spatial information (14). Combining advanced experimental tools, such as using the artificial thymic organoid (ATO) system (48, 49) as an alternative source of T cells, could benefit target validation and TCR discovery.

In summary, IRIS represents a big-data informed computational platform to discover AS-derived cancer immunotherapy targets. In this study, we focused on the application and validation of IRIS for discovering TCR targets. Our results provide experimental evidence for the immunogenicity of AS-derived epitopes and suggest their potential for therapy development. The IRIS software can be downloaded from https://github.com/Xinglab/IRIS

### Materials and Methods

### IRIS Module for RNA-Seq Data Processing.

IRIS accepts standard formats of raw RNA-seq FASTQ files and/or tab-delimited files of quantified AS events [from rMATS-turbo v4.1.0 (26, 28)] as input data. For raw RNA-seq data, IRIS provides a standalone pipeline that aligns RNA-seq reads to the reference human genome, quantifies gene expression, and characterizes AS events. For this paper, the IRIS RNA-seq processing module used the reference human genome hg19 and STAR 2.6.1d (50) two-pass mode for RNA-seq short read alignment. This was followed by quantification of gene expression and AS events using Cufflinks v2.2.1 (51) and rMATS v4.1.0 (rMATS-turbo), respectively, based on the GENCODE (V26) (52) gene annotation utilizing default parameters. AS events characterized by rMATS v4.1.0 contain both annotated and unannotated SJs of known splice sites. To quantify AS events, IRIS generates both PSI (29) values from the rMATS output and SJ read count for every SJ in the alignment file. To remove AS events with low-confidence PSI estimates, low-coverage events are masked as missing values in the output of IRIS-characterized AS events used for downstream analysis. Low-coverage events are defined as events where the sum of read counts of all junctions in the sample of interest is less than 10 (or an average read count less than 10, if filtering by tissue or tumor groups). This pipeline was performed to quantify all common types of AS events, including exon skipping (SE), alternative 3′/5′ splice sites (A3SS/A5SS), and intron retention (RI). The option to identify events from novel splice sites (-novelSS) is also available. This procedure was uniformly applied to all datasets in this study and to the normal and tumor samples for the generation of IRIS DB.

### IRIS DB: A Reference Database of AS Across Normal Human Tissues and Tumor Samples.

IRIS utilizes a reference database of AS profiles from thousands of transcriptomes across normal tissues and tumors to identify AS events with different degrees of tumor association and tumor specificity. Specifically, 9,024 normal samples from the GTEx project (V7) (24) representing 51 normal tissue types of 30 histological sites were uniformly processed as described above. Cell line samples from the GTEx data were excluded from IRIS DB. Additionally, 9,932 TCGA (16, 53) tumor samples were processed to represent 33 tumor types. RNA-seq fastq files for GTEx and TCGA were downloaded from dbGaP and Genomic Data Commons (GDC), respectively. IRIS DB contains ratio-based (PSI) (29) and count-based (SJ read count) quantifications for every AS event detected in a RNA-seq sample in the database. Calculated PSI values and SJ read counts were summarized into indexed reference databases using their coordinates and gene names as keys. Additionally, IRIS DB, along with IRIS utilities to retrieve normal and tumor types from the database to form custom reference panels is made available as a stand-alone resource. In addition, IRIS provides functions for users to build and index their own datasets into custom reference panels.

### IRIS Module for in Silico Screening for Tumor-Associated and -Specific AS Events.

IRIS performs in silico screening to identify tumor-associated and -specific AS events by statistically comparing user-provided RNA-seq data of a given tumor type to a reference panel of user-specified normal or tumor tissues selected from the IRIS DB. Users have the flexibility to specify normal tissues and relevant tumor types to be included in the reference panel. IRIS's in silico screening module provides three distinct screening tests, including ‘tumor-association screen’, ‘tumor-specificity screen’, and ‘tumor-recurrence screen’, to identify AS events of varying degrees of tumor association and specificity.

The default approach, “tumor-association screen”, performs a differential AS analysis between tumor and normal tissues based on the PSI metric. For each event, the differential AS analysis compares the PSI value between a tumor group (user-provided tumor RNA-seq samples) and a normal tissue group (a reference normal tissue type in the IRIS DB) and reports a differential event based on various user-defined criteria, such as p-value and change of PSI value (delta PSI), as well as fold-change (FC) of tumor-enriched isoform (see below paragraphs for detailed definition). Specifically, to define a differential AS event, IRIS sets two default requirements: 1) a significant p-value from a specified statistical test (default: two-sided t-test p<0.01, equal variance disabled), and 2) a threshold of average PSI value difference (default: abs(Δψ)>0.05). The output of ‘tumor-association screen’ will inform the degree of tumor association for each event. This is defined as the number of normal tissue types 1) with significant differential AS from the tumor tissues and 2) same delta PSI direction from the tumor tissues as detected in the tumor-association screen. This degree of tumor association, a key measure for downstream analysis, can be used to define a tumor-associated event by setting a threshold.

‘Tumor-specificity screen’ uses a different approach, which tests for the presence-absence of a given SJ within AS events between tumor and normal tissues and offers a more stringent selection. For each sample group (tumor or normal tissues), IRIS calculates the percentage of samples expressing a given SJ of interest above a user-defined read count threshold. A normal tissue sample with at least 2 reads or a tumor sample with at least 5 reads are considered as samples expressing the SJ. IRIS then determines whether the SJ is expressed in a significantly higher percentage of tumor samples than in normal tissue samples with a significant p-value from a statistical test (default: Fisher exact test p<1.0×10⁻⁶). This step identifies tumor-specific SJs using the number of significant comparisons above a user-defined threshold. Identified tumor-specific SJs are summarized at the event level and subsequently appended to the tumor-association screen output. Finally, IRIS reports a tumor-associated AS event as tumor-specific if it contains one or multiple tumor-specific SJ(s) identified by the tumor-specificity screen that supports the same tumor event.

For each AS event, IRIS defines the ‘tumor-enriched isoform’ as the isoform that is more abundant in tumors than in the tissue-matched normal tissues (or the average of all normal tissues when tissue-matched normal tissues are not specified), which could be either the skipping or inclusion form. Optionally, to rank or filter targets, IRIS estimates the ‘FC of tumor-enriched isoform’, which is calculated as the FC of the proportion of the tumor-enriched isoform in tumor tissues over the proportion of the isoform in tissue-matched normal tissues (or the average proportion of all normal tissue types) of the normal tissue panel.

Default parameters were used for the IRIS analysis for NEPC in this study with minor modifications. For details, please see Target discovery for NEPC using IRIS in below.

### IRIS Module for Predicting AS-Derived TCR and CAR-T Targets.

To obtain peptide sequences of AS-derived tumor isoforms, IRIS translates SJ sequences into amino-acid sequences using known ORFs from the UniProtKB database (31). For each AS event, the SJ peptide sequence of the tumor isoform is compared to the alternative normal isoform to ensure that the SJ of the tumor isoform produces a distinct peptide. By default, SJ peptides are 21-amino-acids long and centered at the SJ site. The actual length of the SJ peptide varies to accommodate the exon length and the occurrence of stop codons upstream of a SJ. Although not used in the current study, the option to generate peptide sequences using all ORFs is available for analyzing events from novel splice sites.

For TCR target prediction, IRIS employs seq2HLA (54), which uses RNA-seq data to characterize HLA class I alleles for each tumor sample. IRIS then uses the IEDB API (30) predictors to obtain putative HLA binding affinities of candidate epitopes. The IEDB ‘recommended’ mode runs several prediction tools to generate multiple predictions for the binding affinity, which IRIS summarizes as a median IC₅₀ value. By default, a threshold of median (IC₅₀)<500 nM denotes a positive prediction for an AS-derived TCR target.

### Proteo-Transcriptomics Data Integration for MS Validation.

IRIS includes an optional proteo-transcriptomics data integration function that incorporates various types of MS data, such as whole-cell proteomics, surfaceomics, and immunopeptidomics data, to validate RNA-seq-based target discovery at the protein level (FIG. 2). Specifically, sequences of AS-derived peptides are added to canonical and isoform sequences of the reference human proteome (downloaded from UniProtKB in September 2018). For immunopeptidomics data, fragment MS spectra are searched against the RNA-seq-based custom proteome library with no enzyme specificity using MSGF+ (55) with the search length limited to 7-15 amino acids. The target-decoy approach is employed to control the false discovery rate (FDR) or ‘QValue’ at 5%.

### IRIS Analysis of Immunopeptidomics Data.

Matching RNA-seq and MS immunopeptidomics data of B lymphoblastoid cell lines (B-LCLs) from two individual donors, B-LCL-S1 and B-LCL-S2, were retrieved from Laumont et al. (32) (GEO: GSM1641206, GSM1641207, and PRIDE: PXD001898). RNA-seq data of the JeKo-1 lymphoma cell line were obtained from the Cancer Cell Line Encyclopedia via the NCI Genomic Data Commons (https://portal.gdc.cancer.gov/legacy-archive/). Corresponding MS immunopeptidomics data of JeKo-1 were retrieved from Khodadoust et al. (33) (PRIDE: PXD004746).

RNA-seq data of normal (B-LCL-S1, B-LCL-S2) and cancer (JeKo-1) cell lines were analyzed by IRIS as described above with minor modifications. Specifically, AS events identified by the IRIS RNA-seq data processing module (with STAR v2.5.3a and rMATS v4.0.2) were not subjected to the in silico screening module, but instead were directly used for the MS search. For MSGF+, FDR was set at 5% for FIGS. 2b and 2e. For the comparison of AS-derived peptides with high and low predicted HLA binding affinities (FIG. 2d), a set of low-affinities peptides was created by randomly selecting peptides with median (IC₅₀)≥500 nM to the same number of total number of high-affinity peptides (median (IC₅₀)<500 nM). Transcript expression level was approximated by taking the product of the gene expression level (FPKM) of the AS event gene and the PSI or 1-PSI value for inclusion or skipping events.

### NEPC RNA-Seq Data.

RNA-seq data of NEPC samples were obtained from a Beltran et al. study (accession no. phs000909) (56) and a Stand Up To Cancer study (accession no. phs000915) (57). Fastq files were downloaded from the database of Genotypes and Phenotypes (dbGAP). In total, 23 NEPC samples are included in this study. To compare SRRM4 gene expression levels across related tumor types, RNA-seq data of CRPC samples were obtained from the Beltran et al. study (accession no. phs000909), the Robinson et al. study (accession no. phs000673) (58), and the Stand-Up-To-Cancer study (accession no. phs000915). RNA-Seq data for PRAD samples were downloaded as part of the TCGA data for IRIS DB from GDC via gdc-client (59). Splicing factor (36) gene expression quantification was completed using FeatureCounts v2.0.1 (60) followed by DESeq2 v1.26.0 (61) normalization.

### Target Discovery for NEPC Using IRIS.

Default screening parameters were used to perform both tumor-association and tumor-specificity screens. For the reference panel, the normal tissue panel was comprised of 11 normal tissue types selected from the IRIS DB, including heart, blood, lung, liver, brain, nerve, muscle, spleen, thyroid, skin, and kidney. Due to the small cell phenotype of NEPC, no tissue-matched normal tissues or related tumor types were specified for the reference panel. Therefore, IRIS did not perform the tumor-recurrence screen in NEPC. The threshold for determining tumor AS events was set to at least eight significant comparisons against the 11 groups from the normal tissue panel in the consistent direction. For each differential test, the minimum number of samples with non-missing values from the NEPC group was set to three, and the equal variance option was enabled in the t-test. For TCR target discovery, two common HLA types, HLA-A*02:01 and HLA-A*03:01, were used for prediction. Default parameters for TCR target prediction were applied.

### Target Selection Criteria for Experimental Validation.

From the pool of 1,651 IRIS prioritized tumor-associated TCR epitopes, a subset of candidates was carried forward for experimental validation due to the throughput limit. We applied three criteria in addition to the formerly described parameters for the NEPC screens: 1) restriction to HLA-A*02:01; 2) exclusion of low FC of tumor-enriched isoform value (FC<2); and 3) exclusion of targets with low gene expression (average FPKM<20). We obtained in 164 unique epitopes that met our criteria. From these epitopes, and an additional 52 epitopes derived from events that contain tumor-specific SJ(s), we selected 76 epitopes to test for immunogenicity and T-cell recognition.

### Code Availability

The IRIS source code is accessible on GitHub at https://github.com/Xinglab/IRIS. A web-based interface is available for generating plots based on tumor-association and -specificity screens. It can be accessed via the IRIS Explorer (https://xingshiny2.research.chop.edu/shiny/IRIS/) under the ‘Browse Data’ tab.

### Data Availability

The RNA-seq data of 23 NEPC samples were retrieved from the Beltran et al. study (56) (accession no. phs000909) and Stand Up To Cancer study (accession no. phs000915). RNA-seq data used to construct IRIS's normal and tumor reference panels of AS events are available from the GTEx project (https://gtexportal.org/) and The Cancer Genome Atlas (TCGA) (https://portal.gdc.gov/legacy-archive/). For the IRIS proteo-transcriptomics analysis, matching RNA-seq data and MS immunopeptidomics data of B-LCL-S1 and B-LCL-S2 cell lines were retrieved from Laumont et al. (GEO: GSM1641206, GSM1641207 and PRIDE: PXD001898). RNA-seq data of the JeKo-1 lymphoma cell line were obtained from the Cancer Cell Line Encyclopedia via the NCI Genomic Data Commons (https://portal.gdc.cancer.gov/legacy-archive/). Corresponding MS immunopeptidomics data of JeKo-1 were retrieved from Khodadoust et al. (PRIDE: PXD004746). For SRRM4 gene expression comparison analysis, RNA-seq data of CRPC samples were obtained from Beltran et al. study (accession no. phs000909), Robinson et al. study (accession no. phs000673), and Stand-Up-To-Cancer study (accession no. phs000915).

### Table A: TCR α/β Polynucleotide and Polypeptide Sequences

The following disclosure provides small cell cancer-enhanced splicing peptide epitopes recognized by TCRs when associated with a human leukocyte antigen, and illustrative polynucleotide sequences of such TCR embodiments of the invention (and the variable region TCR protein sequences that encoded by these polynucleotide sequences).

1.
JPTCR_4
Peptide epitope (Antigen):
(SEQ ID NO: 1)
SLAIGGVTEA
Alpha Chain Nucleotide Sequences:
(SEQ ID NO: 9)
CTTGCTAAGACCACCCAGCCCATCTCCATGGACTCATATGAAGGACAAGA
AGTGAACATAACCTGTAGCCACAACAACATTGCTACAAATGATTATATCA
CGTGGTACCAACAGTTTCCCAGCCAAGGACCACGATTTATTATTCAAGGA
TACAAGACAAAAGTTACAAACGAAGTGGCCTCCCTGTTTATCCCTGCCGA
CAGAAAGTCCAGCACTCTGAGCCTGCCCCGGGTTTCCCTGAGCGACACTG
CTGTGTACTACTGCCTCGTGGGTTGGTTCTCTGGTGGCTACAATAAGCTG
ATTTTTGGAGCAGGGACCAGGCTGGCTGTACACCCATAT
Beta Chain Nucleotide Sequences:
(SEQ ID NO: 10)
GGTGCTGTCGTCTCTCAACATCCGAGCAGGGTTATCTGTAAGAGTGGAAC
CTCTGTGAAGATCGAGTGCCGTTCCCTGGACTTTCAGGCCACAACTATGT
TTTGGTATCGTCAGTTCCCGAAACAGAGTCTCATGCTGATGGCAACTTCC
AATGAGGGCTCCAAGGCCACATACGAGCAAGGCGTCGAGAAGGACAAGTT
TCTCATCAACCATGCAAGCCTGACCTTGTCCACTCTGACAGTGACCAGTG
CCCATCCTGAAGACAGCAGCTTCTACATCTGCAGCGCTAGGGGGGCAGGA
CAAGAGACCCAGTACTTCGGGCCAGGCACGCGGCTCCTGGTGCTCGAGGA
CCT
Alpha chain amino acid sequences:
(SEQ ID NO: 11)
LAKTTQPISMDSYEGQEVNITCSHNNIATNDYITWYQQFPSQGPRFIIQG
YKTKVTNEVASLFIPADRKSSTLSLPRVSLSDTAVYYCLVGWFSGGYNKL
IFGAGTRLAVHPY
Beta chain amino acid sequences:
(SEQ ID NO: 12)
GAVVSQHPSRVICKSGTSVKIECRSLDFQATTMFWYRQFPKQSLMLMATS
NEGSKATYEQGVEKDKFLINHASLTLSTLTVTSAHPEDSSFYICSARGAG
QETQYFGPGTRLLVLEDL
2.
JPTCR_13
Peptide epitope (Antigen):
(SEQ ID NO: 2)
LLLGIAKLLKV
Alpha Chain Nucleotide Sequences:
(SEQ ID NO: 13)
gctcagacagtcactcagtctcaaccagagatgtctgtgcaggaggcaga
gaccgtgaccctgagctgcacatatgacaccagtgagagtgattattatt
tattctggtacaagcagcctcccagcaggcagatgattctcgttattcgc
caagaagcttataagcaacagaatgcaacagagaatcgtttctctgtgaa
cttccagaaagcagccaaatccttcagtctcaagatctcagactcacagc
tgggggatgccgcgatgtatttctgtgcttataggagcgattccgggtat
gcactcaacttcggcaaaggcacctcgctgttggtcacaccccat
Beta Chain Nucleotide Sequences:
(SEQ ID NO: 14)
gctgctggagtcatccagtccccaagacatctgatcaaagaaaagaggga
aacagccactctgaaatgctatcctatccctagacacgacactgtctact
ggtaccagcagggtccaggtcaggacccccagttcctcatttcgttttat
gaaaagatgcagagcgataaaggaagcatccctgatcgattctcagctca
acagttcagtgactatcattctgaactgaacatgagctccttggagctgg
gggactcagccctgtacttctgtgccagcagccccgggactagcgggggg
acagatacgcagtattttggcccaggcacccggctgacagtgctcgGACC
T
Alpha chain amino acid sequences:
(SEQ ID NO: 15)
AQTVTQSQPEMSVQEAETVTLSCTYDTSESDYYLFWYKQPPSRQMILVIR
QEAYKQQNATENRFSVNFQKAAKSFSLKISDSQLGDAAMYFCA
Beta chain amino acid sequences:
(SEQ ID NO: 16)
AAGVIQSPRHLIKEKRETATLKCYPIPRHDTVYWYQQGPGQDPQFLISFY
EKMQSDKGSIPDRFSAQQFSDYHSELNMSSLELGDSALYFCASSPGTSGG
TDTQYFGPGTRLTVLGP
3.
JPTCR_22
Peptide epitope (Antigen):
(SEQ ID NO: 3)
LLAEQPDQV
Alpha Chain Nucleotide Sequences:
(SEQ ID NO: 17)
cagaaggaggtggagcagaattctggacccctcagtgttccagagggagc
cattgcctctctcaactgcacttacagigaccgaggttcccagtccttct
tctggtacagacaatattctgggaaaagccctgagttgataatgtccata
tactccaatggtgacaaagaagatggaaggtttacagcacagctcaataa
agccagccagtatgtttctctgctcatcagagactcccagcccagtgatt
cagccacctacctctgtgccgtgaaacggtggtcaggaggaggtgctgac
ggactcacctttggcaaagggactcatctaatcatccagccctat
Beta Chain Nucleotide Sequences:
(SEQ ID NO: 18)
gatgccatggtcatccagaacccaagataccaggttacccagtttggaaa
gccagtgaccctgagttgttctcagactttgaaccataacgtcatgtact
ggtaccagcagaagtcaagtcaggccccaaagctgctgttccactactat
gacaaagattttaacaatgaagcagacacccctgataacttccaatccag
gaggccgaacacttctttctgctttcttgacatccgctcaccaggcctgg
gggacgcagccatgtacctgtgtgccaccagcagagttcggggcactgaa
gctttctttggacaaggcaccagactcacagttgtagagGACCT
Alpha chain amino acid sequences:
(SEQ ID NO: 19)
QKEVEQNSGPLSVPEGAIASLNCTYSDRGSQSFFWYRQYSGKSPELIMSI
YSNGDKEDGRFTAQLNKASQYVSLLIRDSQPSDSATYLCAVKRWSGGGAD
GLTFGKGTHLIIQPY
Beta chain amino acid sequences:
(SEQ ID NO: 20)
DAMVIQNPRYQVTQFGKPVTLSCSQTLNHNVMYWYQQKSSQAPKLLFHYY
DKDFNNEADTPDNFQSRRPNTSFCFLDIRSPGLGDAAMYLCATSRVRGTE
AFFGQGTRLTVVEDL
4.
JPTCR_45
Peptide epitope (Antigen):
(SEQ ID NO: 4)
STMYYLWML
Alpha Chain Nucleotide Sequences:
(SEQ ID NO: 21)
ggacaaagccttgagcagccctctgaagtgacagctgtggaaggagccat
tgtccagataaactgcacgtaccagacatctgggttttatgggctgtcct
ggtaccagcaacatgatggcggagcacccacatttctttcttacaatggt
ctggatggtttggaggagacaggtcgtttttcttcattccttagtcgctc
tgatagttatggttacctccttctacaggagctccagatgaaagactctg
cctcttacttctgcgctgtagacaccaatgcaggcaaatcaacctttggg
gatgggactacgctcactgtgaagccaaAT
Beta Chain Nucleotide Sequences:
(SEQ ID NO: 22)
gaagcccaagtgacccagaacccaagatacctcatcacagtgactggaaa
gaagttaacagtgacttgttctcagaatatgaaccatgagtatatgtcct
ggtatcgacaagacccagggctgggcttaaggcagatctactattcaatg
aatgttgaggtgactgataagggagatgttcctgaagggtacaaagtctc
tcgaaaagagaagaggaatttccccctgatcctggagtcgcccagcccca
accagacctctctgtacttctgtgccagcagtacttccgggacagggcta
tcgtcacccctccactttgggaacgggaccaggctcactgtgacagAGGA
CCT
Alpha chain amino acid sequences:
(SEQ ID NO: 23)
GQSLEQPSEVTAVEGAIVQINCTYQTSGFYGLSWYQQHDGGAPTFLSYNG
LDGLEETGRFSSFLSRSDSYGYLLLQELQMKDSASYFCAVDTNAGKSTFG
DGTTLTVKPN
Beta chain amino acid sequences:
(SEQ ID NO: 24)
EAQVTQNPRYLITVTGKKLTVTCSQNMNHEYMSWYRQDPGLGLRQIYYSM
NVEVTDKGDVPEGYKVSRKEKRNFPLILESPSPNQTSLYFCASSTSGTGL
SSPLHFGNGTRLTVTEDL
5.
JPTCR_47
Peptide epitope (Antigen):
(SEQ ID NO: 4)
STMYYLWML
Alpha Chain Nucleotide Sequences:
(SEQ ID NO: 25)
cagaaggaggtggagcagaattctggacccctcagtgttccagagggagc
cattgcctctctcaactgcacttacagtgaccgaggttcccagtccttct
tctggtacagacaatattctgggaaaagccctgagttgataatgtccata
tactccaatggtgacaaagaagatggaaggtttacagcacagctcaataa
agccagccagtatgtttctctgctcatcagagactcccagcccagtgatt
cagccacctacctctgtgccgtgcccgctggtggtactagctatggaaag
ctgacatttggacaagggaccatcttgactgtccatccaaAT
Beta Chain Nucleotide Sequences:
(SEQ ID NO: 26)
aaggctggagtcactcaaactccaagatatctgatcaaaacgagaggaca
gcaagtgacactgagctgctcccctatctctgggcataggagtgtatcct
ggtaccaacagaccccaggacagggccttcagttcctctttgaatacttc
agtgagacacagagaaacaaaggaaacttccctggtcgattctcagggcg
ccagttctctaactctcgctctgagatgaatgtgagcaccttggagctgg
gggactcggccctttatctttgcgccagcagcccgacagggactgaagct
ttctttggacaaggcaccagactcacagttgtagAGGACCT
Alpha chain amino acid sequences:
(SEQ ID NO: 27)
QKEVEQNSGPLSVPEGAIASLNCTYSDRGSQSFFWYRQYSGKSPELIMSI
YSNGDKEDGRFTAQLNKASQYVSLLIRDSQPSDSATYLCAVPAGGTSYGK
LTFGQGTILTVHPN
Beta chain amino acid sequences:
(SEQ ID NO: 28)
KAGVTQTPRYLIKTRGQQVTLSCSPISGHRSVSWYQQTPGQGLQFLFEYF
SETQRNKGNFPGRFSGRQFSNSRSEMNVSTLELGDSALYLCASSPTGTEA
FFGQGTRLTVVEDL
6.
JPTCR_50
Peptide epitope (Antigen):
(SEQ ID NO: 4)
STMYYLWML
Alpha Chain Nucleotide Sequences:
(SEQ ID NO: 29)
aaacaggaggtgacgcagattcctgcagctctgagtgtcccagaaggaga
aaacttggttctcaactgcagtttcactgatagcgctatttacaacctcc
agtggtttaggcaggaccctgggaaaggtctcacatctctgttgcttatt
cagtcaagtcagagagagcaaacaagtggaagacttaatgcctcgctgga
taaatcatcaggacgtagtactttatacattgcagcttctcagcctggtg
actcagccacctacctctgtgtgtgacccgtattaacgactacaagctca
gctttggagccggaaccacagtaactgtaagagcaaAT
Beta Chain Nucleotide Sequences:
(SEQ ID NO: 30)
aaggctggagtcactcaaactccaagatatctgatcaaaacgagaggaca
gcaagtgacactgagctgctcccctatctctgggcataggagtgtatcct
ggtaccaacagaccccaggacagggccttcagttcctctttgaatacttc
agtgagacacagagaaacaaaggaaacttccctggtcgattctcagggcg
ccagttctctaactctcgctctgagatgaatgtgagcaccttggagctgg
gggactcggccctttatctttgcgccagctcaatgaacactgaagctttc
tttggacaaggcaccagactcacagttgtagAGGACCT
Alpha chain amino acid sequences:
(SEQ ID NO: 31)
KQEVTQIPAALSVPEGENLVLNCSFTDSAIYNLQWFRQDPGKGLTSLLLI
QSSQREQTSGRLNASLDKSSGRSTLYIAASQPGDSATYLCAVTRINDYKL
SFGAGTTVTVRAN
Beta chain amino acid sequences:
(SEQ ID NO: 32)
KAGVTQTPRYLIKTRGQQVTLSCSPISGHRSVSWYQQTPGQGLQFLFEYF
SETQRNKGNFPGRFSGRQFSNSRSEMNVSTLELGDSALYLCASSMNTEAF
FGQGTRLTVVEDL
7.
JPTCR_52
Peptide epitope (Antigen):
(SEQ ID NO: 6)
FLSELEPPA
Alpha Chain Nucleotide Sequences:
(SEQ ID NO: 33)
gcccagtctgtgagccagcataaccaccacgtaattctctctgaagcagc
ctcactggagttgggatgcaactattcctatggtggaactgttaatctct
tctggtatgtccagtaccctggtcaacaccttcagcttctcctcaagtac
ttttcaggggatccactggttaaaggcatcaagggctttgaggctgaatt
tataaagagtaaattctcctttaatctgaggaaaccctctgtgcagtgga
gtgacacagctgagtacttctgtgccgcgtataacaccgacaagctcatc
tttgggactgggaccagattacaagtctttccaaAT
Beta Chain Nucleotide Sequences:
(SEQ ID NO: 34)
aaggctggagtcactcaaactccaagatatctgatcaaaacgagaggaca
gcaagtgacactgagctgctcccctatctctgggcataggagtgtatcct
ggtaccaacagaccccaggacagggccttcagttcctctttgaatacttc
agtgagacacagagaaacaaaggaaacttccctggtcgattctcagggcg
ccagttctctaactctcgctctgagatgaatgtgagcaccttggagctgg
gggactcggccctttatctttgcgccagcagcggcgcggggggaccccaa
gagacccagtacttcgggccaggcacgcggctcctggtgctcgAGGACCT
Alpha chain amino acid sequences:
(SEQ ID NO: 35)
AQSVSQHNHHVILSEAASLELGCNYSYGGTVNLFWYVQYPGQHLQLLLKY
FSGDPLVKGIKGFEAEFIKSKFSFNLRKPSVQWSDTAEYFCAAYNTDKLI
FGTGTRLQVFPN
Beta chain amino acid sequences:
(SEQ ID NO: 36)
KAGVTQTPRYLIKTRGQQVTLSCSPISGHRSVSWYQQTPGQGLQFLFEYF
SETQRNKGNFPGRFSGRQFSNSRSEMNVSTLELGDSALYLCASSGAGGPQ
ETQYFGPGTRLLVLEDL
8.
JPTCR_56
Peptide epitope (Antigen):
(SEQ ID NO: 4)
STMYYLWML
Alpha Chain Nucleotide Sequences:
(SEQ ID NO: 37)
cagaaggaggtggagcaggatcctggaccactcagtgttccagagggagc
cattgtttctctcaactgcacttacagcaacagtgcttttcaatacttca
tgtggtacagacagtattccagaaaaggccctgagttgctgatgtacaca
tactccagtggtaacaaagaagatggaaggtttacagcacaggtcgataa
atccagcaagtatatctccttgttcatcagagactcacagcccagtgatt
cagccacctacctctgtgcaatgacaggaaacacacctcttgtctttgga
aagggcacaagactttctgtgattgcaaAT
Beta Chain Nucleotide Sequences:
(SEQ ID NO: 38)
ggtgctgtcgtctctcaacatccgagcagggttatctgtaagagtggaac
ctctgtgaagatcgagtgccgttccctggactttcaggccacaactatgt
tttggtatcgtcagttcccgaaacagagtctcatgctgatggcaacttcc
aatgagggctccaaggccacatacgagcaaggcgtcgagaaggacaagtt
tctcatcaaccatgcaagcctgaccttgtccactctgacagtgaccagtg
cccatcctgaagacagcagcttctacatctgcagtgctttacagggcgct
gaagctttctttggacaaggcaccagactcacagttgtagAGGACCT
Alpha chain amino acid sequences:
(SEQ ID NO: 39)
QKEVEQDPGPLSVPEGAIVSLNCTYSNSAFQYFMWYRQYSRKGPELLMYT
YSSGNKEDGRFTAQVDKSSKYISLFIRDSQPSDSATYLCAMTGNTPLVFG
KGTRLSVIAN
Beta chain amino acid sequences:
(SEQ ID NO: 40)
GAVVSQHPSRVICKSGTSVKIECRSLDFQATTMFWYRQFPKQSLMLMATS
NEGSKATYEQGVEKDKFLINHASLTLSTLTVTSAHPEDSSFYICSALQGA
EAFFGQGTRLTVVEDL
9.
JPTCR_128
Peptide epitope (Antigen):
(SEQ ID NO: 7)
FDLAYGSVITV
Alpha Chain Nucleotide Sequences:
(SEQ ID NO: 41)
ggagagagtgtggggctgcatcttcctaccctgagtgtccaggagggtga
caactctattatcaactgtgcttattcaaacagcgcctcagactacttca
tttggtacaagcaagaatctggaaaaggtcctcaattcattatagacatt
cgttcaaatatggacaaaaggcaaggccaaagagtcaccgttttattgaa
taagacagtgaaacatctctctctgcaaattgcagctactcaacctggag
actcagctgtctacttttgtgcagagaggggtggtactagctatggaaag
ctgacatttggacaagggaccatcttgactgtccatccaaat
Beta Chain Nucleotide Sequences:
(SEQ ID NO: 42)
gacacagctgtttcccagactccaaaatacctggtcacacagatgggaaa
cgacaagtccattaaatgtgaacaaaatctgggccatgatactatgtatt
ggtataaacaggactctaagaaatttctgaagataatgtttagctacaat
aataaggagctcattataaatgaaacagttccaaatcgcttctcacctaa
atctccagacaaagctcactta
Alpha chain amino acid sequences:
(SEQ ID NO: 43)
GESVGLHLPTLSVQEGDNSIINCAYSNSASDYFIWYKQESGKGPQFIIDI
RSNMDKRQGQRVTVLLNKTVKHLSLQIAATQPGDSAVYFCAERGGTSYGK
LTFGQGTILTVHPN
Beta chain amino acid sequences:
(SEQ ID NO: 44)
DTAVSQTPKYLVTQMGNDKSIKCEQNLGHDTMYWYKQDSKKFLKIMFSYN
NKELIINETVPNRFSPKSPDKAHL
10.
JPTCR_188
Peptide epitope (Antigen):
(SEQ ID NO: 7)
FDLAYGSVITV
Alpha Chain Nucleotide Sequences:
(SEQ ID NO: 45)
cagaaggaggtggagcagaattctggacccctcagtgttccagagggagc
cattgcctctctcaactgcacttacagtgaccgaggttcccagtccttct
tctggtacagacaatattctgggaaaagccctgagttgataatgtccata
tactccaatggtgacaaagaagatggaaggtttacagcacagctcaataa
agccagccagtatgtttctctgctcatcagagactcccagcccagtgatt
cagccacctacctctgtgccgtgaatcatcagggagcccagaagctggta
tttggccaaggaaccaggctgactatcaacccaaat
Beta Chain Nucleotide Sequences:
(SEQ ID NO: 46)
gattctggagtcacacaaaccccaaagcacctgatcacagcaactggaca
gcgagtgacgctgagatgctcccctaggtctggagacctctctgtgtact
ggtaccaacagagcctggaccagggcctccagttcctcattcaglattat
aalggagaagagagagcaaaaggaaacattcttgaacgattctccgcaca
acagttccctgacttgcactctgaactaaacctgagctctctggagctgg
gggactcagctttgtatttctgtgccagcagcgtggggacccttaatgag
cagttcttcgggccagggacacggctcaccgtgctagAGGACCTG
Alpha chain amino acid sequences:
(SEQ ID NO: 47)
QKEVEQNSGPLSVPEGAIASLNCTYSDRGSQSFFWYRQYSGKSPELIMSI
YSNGDKEDGRFTAQLNKASQYVSLLIRDSQPSDSATYLCAVNHQGAQKLV
FGQGTRLTINPN
Beta chain amino acid sequences:
(SEQ ID NO: 48)
DSGVTQTPKHLITATGQRVTLRCSPRSGDLSVYWYQQSLDQGLQFLIQYY
NGEERAKGNILERFSAQQFPDLHSELNLSSLELGDSALYFCASSVGTLNE
QFFGPGTRLTVLEDL
11.
JPTCR_208
Peptide epitope (Antigen):
(SEQ ID NO: 7)
FDLAYGSVITV
Alpha Chain Nucleotide Sequences:
(SEQ ID NO: 49)
acccagctgctggagcagagccctcagtttctaagcatccaagagggaga
aaatctcactgtgtactgcaactcctcaagtgttttttccagcttacaat
ggtacagacaggagcctggggaaggtcctgtcctcctggtgacagtagtt
acgggtggagaagtgaagaagctgaagagactaacctttcagtttggtga
tgcaagaaaggacagttctctccacatcactgcggcccagcctggtgata
caggcctctacctctgtgcaggagtaccaggaggaagctacatacctaca
tttggaagaggaaccagccttattgttcatccgtat
Beta Chain Nucleotide Sequences:
(SEQ ID NO: 50)
gacacagctgtttcccagactccaaaatacctggtcacacagatgggaaa
cgacaagtccattaaatgtgaacaaaatctgggccatgatactatgtatt
ggtataaacaggactctaagaaatttctgaagataatgtttagctacaat
aataaggagctcattataaatgaaacagttccaaatcgcttctcacctaa
atctccagacaaagctcacttaaatcttcacatcaattccctggagcttg
gtgactctgctgtgtatttctgtgccagcagccaggtcgagacggaagga
gctaactatggctacaccttcggttcggggaccaggttaaccgttgtagA
GGACCTG
Alpha chain amino acid sequences:
(SEQ ID NO: 52)
TQLLEQSPQFLSIQEGENLTVYCNSSSVFSSLQWYRQEPGEGPVLLVTVV
TGGEVKKLKRLTFQFGDARKDSSLHITAAQPGDTGLYLCAGVPGGSYIPT
FGRGTSLIVHPY
Beta chain amino acid sequences:
(SEQ ID NO: 53)
DTAVSQTPKYLVTQMGNDKSIKCEQNLGHDTMYWYKQDSKKFLKIMFSYN
NKELIINETVPNRFSPKSPDKAHLNLHINSLELGDSAVYFCASSQVETEG
ANYGYTFGSGTRLTVVEDL
12.
JPTCR_238
Peptide epitope (Antigen):
(SEQ ID NO: 8)
SLDGTTTKA
Alpha Chain Nucleotide sequences:
(SEQ ID NO: 54)
gctcagaaggtaactcaagcgcagactgaaatttctgtggtggagaagga
ggatgtgaccttggactgtgtgtatgaaacccgtgatactacttattact
tattctggtacaagcaaccaccaagtggagaattggttttccttattcgt
cggaactcttttgatgagcaaaatgaaataagtggtcggtattcttggaa
cttccagaaatccaccagttccttcaacttcaccatcacagcctcacaag
tcgtggactcagcagtatacttctgtgctctgagtggtggaggcttcaaa
actatctttggagcaggaacaagactatttgttaaagcaaat
Beta Chain Nucleotide sequences:
(SEQ ID NO: 55)
tctcagactattcatcaatggccagcgaccctggtgcagcctgtgggcag
cccgctctctctggagtgcactgtggagggaacatcaaaccccaacctat
actggtaccgacaggctgcaggcaggggcctccagctgctcttctactcc
gttggtattggccagatcagctctgaggtgccccagaatctctcagcctc
cagaccccaggaccggcagttcatcctgagttctaagaagctccttctca
gtgactctggcttctatctctgtgcctggaacggacagggctcctacaat
gagcagttcttcgggccagggacacggctcaccgtgctagAGGACCTg
Alpha chain amino acid sequences:
(SEQ ID NO: 56)
AQKVTQAQTEISVVEKEDVTLDCVYETRDTTYYLFWYKQPPSGELVFLIR
RNSFDEQNEISGRYSWNFQKSTSSFNFTITASQVVDSAVYFCALSGGGFK
TIFGAGTRLFVKAN
Beta chain amino acid sequences:
(SEQ ID NO: 57)
SQTIHQWPATLVQPVGSPLSLECTVEGTSNPNLYWYRQAAGRGLQLLFYS
VGIGQISSEVPQNLSASRPQDRQFILSSKKLLLSDSGFYLCAWNGQGSYN
EQFFGPGTRLTVLEDL

### PUBLICATIONS

All publications mentioned herein (e.g. those listed numerically herein) are incorporated herein by reference to disclose and describe the methods and/or materials in connection with which the publications are cited. Publications cited herein are cited for their disclosure prior to the filing date of the present application. Nothing here is to be construed as an admission that the inventors are not entitled to antedate the publications by virtue of an earlier priority date or prior date of invention. Further, the actual publication dates may be different from those shown and require independent verification. The following references include descriptions of methods and materials in this field of technology.

### REFERENCES

1. C. Sun, R. Mezzadra, T. N. Schumacher, Regulation and Function of the PD-L1 Checkpoint. Immunity 48, 434-452 (2018).
2. S. A. Rosenberg, N. P. Restifo, Adoptive cell transfer as personalized immunotherapy for human cancer. Science (80-.). 348, 62-68 (2015).
3. T. N. Schumacher, R. D. Schreiber, Neoantigens in cancer immunotherapy. Science (80-.). 348, 69-74 (2015).
4. M. Yarchoan, B. A. Johnson, E. R. Lutz, D. A. Laheru, E. M. Jaffee, Targeting neoantigens to augment antitumour immunity. Nat. Rev. Cancer 17, 209-222 (2017).
5. P. G. Coulie, B. J. Van Den Eynde, P. Van Der Bruggen, T. Boon, Tumour antigens recognized by T lymphocytes: At the core of cancer immunotherapy. Nat. Rev. Cancer 14, 135-146 (2014).
6. C. H. Lee, R. Yelensky, K. Jooss, T. A. Chan, Update on Tumor Neoantigens and Their Utility: Why It Is Good to Be Different. Trends Immunol. 39, 536-548 (2018).
7. A. Vitiello, M. Zanetti, Neoantigen prediction and the need for validation. Nat. Biotechnol. 2017 359 35, 815-817 (2017).
8. The problem with neoantigen prediction. Nat. Biotechnol. 2017 352 35, 97-97 (2017).
9. R. Marty, et al., MHC-I Genotype Restricts the Oncogenic Mutational Landscape. Cell 171, 1272-1283.e15 (2017).
10. P. A. Ott, et al., An immunogenic personal neoantigen vaccine for patients with melanoma. Nature 547, 217-221 (2017).
11. R. Nemecek, et al., Personalized RNA mutanome vaccines mobilize poly-specific therapeutic immunity against cancer. Nature 547, 222-226 (2017).
12. B. M. Carreno, et al., Cancer immunotherapy. A dendritic cell vaccine increases the breadth and diversity of melanoma neoantigen-specific T cells. Science (80-.). 348, 803-808 (2015).
13. S. C. Bonnal, I. López-Oreja, J. Valcárcel, Roles and mechanisms of alternative splicing in cancer—implications for care. Nat. Rev. Clin. Oncol. 17, 457-474 (2020).
14. Y. Pan, et al., RNA Dysregulation: An Expanding Source of Cancer Immunotherapy Targets. Trends Pharmacol. Sci. 42, 268-282 (2021).
15. S. Cherry, K. W. Lynch, Alternative splicing and cancer: Insights, opportunities, and challenges from an expanding view of the transcriptome. Genes Dev. 34, 1005-1016 (2020).
16. A. Kahles, et al., Comprehensive Analysis of Alternative Splicing Across Tumors from 8,705 Patients. Cancer Cell 34, 211-224.e6 (2018).
17. A. C. Smart, et al., Intron retention is a source of neoepitopes in cancer. Nat. Biotechnol. 36, 1056 (2018).
18. C. M. Laumont, et al., Noncoding regions are the main source of targetable tumor-specific antigens. Sci. Transl. Med. 10 (2018).
19. T. Y. Wang, et al., A pan-cancer transcriptome analysis of exitron splicing identifies novel cancer driver genes and neoepitopes. Mol. Cell 81, 2246-2260.e12 (2021).
20. M. Zhang, et al., RNA editing derived epitopes function as cancer antigens to elicit immune responses. Nat. Commun. 2018 919, 1-10 (2018).
21. L. Frankiw, D. Baltimore, G. Li, Alternative mRNA splicing in cancer immunotherapy (Nature Publishing Group, 2019).
22. Z. Zhang, et al., ASNEO: Identification of personalized alternative splicing based neoantigens with RNA-seq. Aging (Albany NY) 12, 14633 (2020).
23. S. Chai, et al., NeoSplice: A bioinformatics method for prediction of splice variant neoantigens. Bioinforma. Adv., 0-0 (2022).
24. T. Gte. GTEx Consortium, The Genotype-Tissue Expression (GTEx) project. Nat. Genet. 45, 580-5 (2013).
25. J. N. Cancer Genome Atlas Research Network, et al., The Cancer Genome Atlas Pan-Cancer analysis project. Nat. Genet. 45, 1113-20 (2013).
26. J. W. Phillips, et al., Pathway-guided analysis identifies Myc-dependent alternative pre-mRNA splicing in aggressive prostate cancers. Proc. Natl. Acad. Sci. U.S.A 117, 5269-5279 (2020).
27. P. J. Vlachostergios, L. Puca, H. Beltran, Emerging Variants of Castration-Resistant Prostate Cancer. Curr. Oncol. Rep. 19, 1-10 (2017).
28. S. Shen, et al., rMATS: Robust and flexible detection of differential alternative splicing from replicate RNA-Seq data. Proc. Natl. Acad. Sci. U.S.A 111, E5593-E5601 (2014).
29. Y. Katz, E. T. Wang, E. M. Airoldi, C. B. Burge, Analysis and design of RNA sequencing experiments for identifying isoform regulation. Nat. Methods 7, 1009-1015 (2010).
30. R. Vita, et al., The immune epitope database (IEDB) 3.0. Nucleic Acids Res. 43, D405-D412 (2015).
31. T. UniProt Consortium, Erratum: UniProt: the universal protein knowledgebase (Nucleic acids research (2017) 45 D1 (D158-D169)). Nucleic Acids Res. 46, 2699 (2018).
32. C. M. Laumont, et al., Global proteogenomic analysis of human MHC class I-associated peptides derived from non-canonical reading frames. Nat. Commun. 7, 10238 (2016).
33. M. S. Khodadoust, et al., Antigen presentation profiling reveals recognition of lymphoma immunoglobulin neoantigens. Nature 543, 723-727 (2017).
34. J. G. Abelin, et al., Mass Spectrometry Profiling of HLA-Associated Peptidomes in Mono-allelic Cells Enables More Accurate Epitope Prediction. Immunity 46, 315-326 (2017).
35. M. Irimia, et al., A highly conserved program of neuronal microexons is misregulated in autistic brains. Cell 159, 1511-1523 (2014).
36. H. Han, et al., MBNL proteins repress ES-cell-specific alternative splicing and reprogramming. Nature 498 (2013).
37. Y. Li, et al., SRRM4 Drives Neuroendocrine Transdifferentiation of Prostate Adenocarcinoma Under Androgen Receptor Pathway Inhibition. Eur. Urol. 71, 68-78 (2017).
38. T. Gonatopoulos-Pournatzis, et al., Autism-Misregulated eIF4G Microexons Control Synaptic Translation and Higher Order Cognitive Functions. Mol. Cell 77, 1176-1192.e16 (2020).
39. P. A. Nesterenko, et al., HLA-A*02:01 restricted T cell receptors against the highly conserved SARS-COV-2 polymerase cross-react with human coronaviruses. Cell Rep. 37 (2021).
40. M. Wolfl, et al., Activation-induced expression of CD137 permits detection, isolation, and expansion of the full repertoire of CD8+ T cells responding to antigen without requiring knowledge of epitope specificities. Blood 110, 201-210 (2007).
41. M. Wölfl, J. Kuball, M. Eyrich, P. G. Schlegel, P. D. Greenberg, Use of CD137 to study the full repertoire of CD8+ T cells without the need to know epitope specificities. Cytometry. A 73, 1043 (2008).
42. Z. Mao, et al., Physical and in silico immunopeptidomic profiling of a cancer antigen prostatic acid phosphatase reveals targets enabling TCR isolation. Proc. Natl. Acad. Sci. U.S.A 119, e2203410119 (2022).
43. R. A. Morgan, et al., Cancer Regression in Patients After Transfer of Genetically Engineered Lymphocytes. Science 314, 126 (2006).
44. S. Terry, H. Beltran, The many faces of neuroendocrine differentiation in prostate cancer progression. Front. Oncol. 4 March, 60 (2014).
45. E. Park, Z. Pan, Z. Zhang, L. Lin, Y. Xing, The Expanding Landscape of Alternative Splicing Variation in Human Populations. Am. J. Hum. Genet. 102, 11-26 (2018).
46. M. Oka, et al., Aberrant splicing isoforms detected by full-length transcriptome sequencing as transcripts of potential neoantigens in non-small cell lung cancer. Genome Biol. 22, 1-30 (2021).
47. S. L. Amarasinghe, et al., Opportunities and challenges in long-read sequencing data analysis. Genome Biol. 21 (2020).
48. C. S. Seet, et al., Generation of mature T cells from human hematopoietic stem and progenitor cells in artificial thymic organoids. Nat. Methods 14, 521-530 (2017).
49. A. Montel-Hagen, et al., Organoid-Induced Differentiation of Conventional T Cells from Human Pluripotent Stem Cells. Cell Stem Cell 24, 376-389.e8 (2019).
50. A. Dobin, et al., STAR: ultrafast universal RNA-seq aligner. Bioinformatics 29, 15-21 (2013).
51. C. Trapnell, et al., Differential gene and transcript expression analysis of RNA-seq experiments with TopHat and Cufflinks. Nat. Protoc. 7, 562-78 (2012).
52. J. Harrow, et al., GENCODE: The reference human genome annotation for The ENCODE Project. Genome Res. 22, 1760-1774 (2012).
53. J. N. Weinstein, et al., The cancer genome atlas pan-cancer analysis project. Nat. Genet. 45, 1113-1120 (2013).
54. S. Boegel, et al., HLA typing from RNA-Seq sequence reads. Genome Med. 4, 102 (2012).
55. S. Kim, P. A. Pevzner, MS-GF+ makes progress towards a universal database search tool for proteomics. Nat. Commun. 5, 5277 (2014).
56. H. Beltran, et al., Divergent clonal evolution of castration-resistant neuroendocrine prostate cancer. Nat. Med. 22, 298-305 (2016).
57. D. R. Robinson, et al., Integrative clinical genomics of metastatic cancer. Nature 548, 297-303 (2017).
58. D. Robinson, et al., Integrative clinical genomics of advanced prostate cancer. Cell 161, 1215-1228 (2015).
59. R. L. Grossman, et al., Toward a Shared Vision for Cancer Genomic Data. N. Engl. J. Med. 375, 1109-1112 (2016).
60. Y. Liao, G. K. Smyth, W. Shi, featureCounts: an efficient general purpose program for assigning sequence reads to genomic features. Bioinformatics 30, 923-930 (2014).
61. M. I. Love, W. Huber, S. Anders, Moderated estimation of fold change and dispersion for RNA-seq data with DESeq2. Genome Biol. 15, 1-21 (2014).

### CONCLUSION

This concludes the description of the illustrative embodiments of the present invention. The foregoing description of one or more embodiments of the invention has been presented for the purposes of illustration and description. It is not intended to be exhaustive or to limit the invention to the precise form disclosed. Many modifications and variations are possible in light of the above teaching.

1. A composition comprising a polynucleotide disposed in a vector, wherein:
the polynucleotide encodes a Vα T cell receptor polypeptide and/or a Vβ T cell receptor polypeptide; and
when a Vα/Vβ T cell receptor comprising the Vα T cell receptor polypeptide and/or the Vβ T cell receptor polypeptide is expressed in a CD 8⁺ T cell, the Vα/Vβ T cell receptor recognizes a peptide comprising a small cell cancer-enhanced splicing epitope associated with a human leukocyte antigen.

2. The composition of claim 1, wherein the human leukocyte antigen comprises HLA-A*02:01.

3. The composition of claim 1, wherein the peptide comprises an amino acid sequence:
(SEQ ID NO: 1)
SLAIGGVTEA;
(SEQ ID NO: 2)
LLLGIAKLLKV;
(SEQ ID NO: 3)
LLAEQPDQV;
(SEQ ID NO: 4)
STMYYLWML;
(SEQ ID NO: 5)
FLSELEPPA;
(SEQ ID NO: 6)
FDLAYGSVITV;
or
(SEQ ID NO: 7)
SLDGTTTKA

4. The composition of claim 1, wherein the T cell receptor comprises at least one polypeptide sequence having at least a 95% sequence identity to SEQ ID NO: 11, SEQ ID NO: 12, SEQ ID NO: 15, SEQ ID NO: 16, SEQ ID NO: 19, SEQ ID NO: 20, SEQ ID NO: 23, SEQ ID NO: 24, SEQ ID NO: 27, SEQ ID NO: 28, SEQ ID NO: 31, SEQ ID NO: 32, SEQ ID NO: 35, SEQ ID NO: 36, SEQ ID NO: 39, SEQ ID NO: 40, SEQ ID NO: 43, SEQ ID NO: 44, SEQ ID NO: 47, SEQ ID NO: 48, SEQ ID NO: 52, SEQ ID NO: 53, SEQ ID NO: 56, or SEQ ID NO: 57.

5. The composition of claim 4, wherein the T cell receptor comprises at least one polypeptide sequence comprising an amino acid substitution mutation in SEQ ID NO: 11, SEQ ID NO: 12, SEQ ID NO: 15, SEQ ID NO: 16, SEQ ID NO: 19, SEQ ID NO: 20, SEQ ID NO: 23, SEQ ID NO: 24, SEQ ID NO: 27, SEQ ID NO: 28, SEQ ID NO: 31, SEQ ID NO: 32, SEQ ID NO: 35, SEQ ID NO: 36, SEQ ID NO: 39, SEQ ID NO: 40, SEQ ID NO: 43, SEQ ID NO: 44, SEQ ID NO: 47, SEQ ID NO: 48, SEQ ID NO: 52, SEQ ID NO: 53, SEQ ID NO: 56, or SEQ ID NO: 57.

6. The composition of claim 3, wherein the vector is a Sendai viral vector, an adenoviral vector, an adeno-associated virus vector, a retroviral vector, or a lentiviral vector.

7. The composition of claim 4, wherein the vector comprises a polynucleotide encoding a Vα polypeptide in combination with a polynucleotide encoding a Vβ polypeptide disposed in the vector so that a Vα/Vβ T cell receptor (TCR) is expressed on the surface of a CD 8⁺ T cell.

8. A composition of matter comprising a host cell transduced with a vector of claim 1.

9. The composition of claim 8, wherein the host cell is a human CD 8⁺ T cell.

10. The composition of claim 9, wherein the composition is a pharmaceutical composition comprising one more pharmaceutically acceptable excipients selected from the group consisting of buffering agents, antimicrobial agents, tonicity adjusting agents, wetting agents, detergents and pH adjusting agents.

11. The composition of claim 10, wherein:
the CD8⁺ T cell is obtained from an individual diagnosed with a cancer that expresses a small cell cancer-enhanced splicing peptide antigen; and
the CD8⁺ T cell is transduced with a vector comprising a polynucleotide encoding a TCR Vα polypeptide in combination with a polynucleotide encoding a TCR Vβ polypeptide such that a heterologous TCR is expressed on a surface of the CD8⁺ T cell, wherein the heterologous TCR recognizes a small cell cancer-enhanced splicing peptide antigen associated with a human leukocyte antigen expressed on the surface of cells of the cancer.

12. The composition of claim 11, wherein the vector is a retroviral vector.

13. A method of killing cancer cells that express a small cell cancer-enhanced splicing peptide antigen, the method comprising combining the cancer cells with CD8⁺ T cells of claim 9 under conditions that allow a heterologous TCR to be expressed on the surface of the CD8⁺ T cell and recognize small cell cancer-enhanced splicing peptides associated with a human leukocyte antigens expressed on the surface of cells of the cancer, so that the cancer cells are recognized and killed.

14. The method of claim 13, wherein the method is performed in vivo on a patient infused with the CD8⁺ T cells.

15. The method of claim 13, wherein the cancer cells form small cell tumors.

16. The method of claim 13, wherein the cancer cells are small cell prostate cancer cells or small cell lung cancer cells.

17. The method of claim 13, wherein the method comprises administering a first modified CD8⁺ T cell that targets a small cell cancer-enhanced splicing peptide antigen associated with a first human leukocyte antigen human leukocyte antigen in combination with a second CD8⁺ T cell that targets a small cell cancer-enhanced splicing peptide antigen associated with second human leukocyte antigen.

18. The use of the polynucleotide of claim 1 or the CD8⁺ T cell of claim 9 for the manufacture of a medicament for the treatment of a cancer.

19. The use of claim 18, wherein the polynucleotide comprises at least one polynucleotide encoding a polypeptide sequence having at least a 95% sequence identity to SEQ ID NO: 11, SEQ ID NO: 12, SEQ ID NO: 15, SEQ ID NO: 16, SEQ ID NO: 19, SEQ ID NO: 20, SEQ ID NO: 23, SEQ ID NO: 24, SEQ ID NO: 27, SEQ ID NO: 28, SEQ ID NO: 31, SEQ ID NO: 32, SEQ ID NO: 35, SEQ ID NO: 36, SEQ ID NO: 39, SEQ ID NO: 40, SEQ ID NO: 43, SEQ ID NO: 44, SEQ ID NO: 47, SEQ ID NO: 48, SEQ ID NO: 52, SEQ ID NO: 53, SEQ ID NO: 56, or SEQ ID NO: 57.

20. The use of claim 19, wherein the cancer is a small cell prostate cancer or a small cell lung cancer.
